(19) United States
(12) Patent Application Publication
(10) Pub. No.: US 2024/0382112 A1
(43) Pub. Date: Nov. 21, 2024

(54) SYSTEM FOR DETERMINING A PHYSIOLOGICAL PARAMETER OF A SUBJECT

(71) Applicant: UMC Utrecht Holding B.V., Utrecht (NL)

(72) Inventors: Cornelis Antonius Theodorus Van Den Berg, Utrecht (NL); Bart R. Steensma, Utrecht (NL)

(21) Appl. No.: 18/692,795

(22) PCT Filed: Sep. 6, 2022
(86) PCT No.: PCT/EP2022/074751

§ 371 Date: Mar. 15, 2024

(30) Foreign Application Priority Data
Sep. 17, 2021 (EP) 21197565.1

(51) Int. Cl. A61B 5/11; A61B 5/00; A61B 5/05; G01S 13/62; G16H 40/67; H01Q 9/26

(52) U.S. Cl. CPC A61B 5/1126; A61B 5/05; A61B 5/7267; G01S 13/62; G16H 40/67; H01Q 9/265

(57) ABSTRACT

The invention relates to a system 1 for determining a physiological parameter like a stroke volume of the heart of a subject 7. A measurement device includes a) an RF antenna module 3 with one or more RF antennas 4, 5 and b) an RF instrument 2 like a vector network analyzer configured to transmit RF power into the RF antenna module, to receive an RF signal from the RF antenna module and to provide a motion signal that is related to a mechanical movement of a structure within the subject based on the received RF signal. The physiological parameter is determined based on the provided motion signal and a model that provides, as an output, the physiological parameter if, as an input, the motion signal is provided.

### FIELD OF THE INVENTION

The invention relates to a system, method and computer program for determining a physiological parameter of a subject. The invention relates further to a measurement device and a determination device for determining a physiological parameter of a subject, wherein the measurement device is configured to provide a motion signal and the determination device is configured to determine the physiological parameter based on the provided motion signal. The invention also relates to a training system, training method and training computer program for training a model to be used by the determination device for determining the physiological parameter of the subject. The physiological parameter preferentially is a heart-related physiological parameter or a lung-related physiological parameter.

### BACKGROUND OF THE INVENTION

The articles “Wearable radio-frequency sensing of respiratory rate, respiratory volume, and heart rate” by P. Sharma et al., npj Digital Medicine 3, volume 98, pages 1 to 10 (2020) and “Microwave apexcardiography” by J. Lin et al., IEEE T-MTT 6, volume 27, pages 618 to 620 (1979) disclose systems for determining respiratory rate, respiratory volume and heart rate, wherein the systems use wearable radiofrequency (RF) sensing devices for determining these physiological parameters. The used sensors can be over-clothing wearable RF sensors.

### SUMMARY OF THE INVENTION

It is an object of the present invention to provide a system, method and computer program which allow for an improved determination of a physiological parameter of a subject. It is a further object of the present invention to provide a measurement device and a determination device for determining a physiological parameter of the subject, wherein the determination device is configured to allow for an improved determination of the physiological parameter based on a motion signal provided by the measurement device. Moreover, the invention relates to a training system, method and computer program for training a model to be used by the system and the determination device for allowing for the improved determination of the physiological parameter.

In a first aspect of the present invention a system for determining a physiological parameter of a subject is presented, wherein the system comprises:
a measurement device including a) an RF antenna module comprising one or more RF antennas and b) an RF instrument connected to the RF antenna module and configured to transmit RF power into the RF antenna module, to receive an RF signal from the RF antenna module and to provide a motion signal that is related to a mechanical movement of a structure within the subject based on the received RF signal,
a determination device configured to determine the physiological parameter based on the provided motion signal, wherein the determination device comprises a model providing module configured to provide a model that provides, as an output, a physiological parameter if, as an input, a motion signal is provided, and a processor configured to determine the physiological parameter based on the provided model and the provided motion signal.

Since the RF instrument and the RF antenna module are configured to provide a motion signal that is related to a mechanical movement of a structure like an organ within the subject, i.e. since the RF instrument and the RF antenna module are configured such that the measurement region, which is the region in which the measurement device can sense mechanical motion, covers the structure, the mechanical movement of the structure within the subject directly influences the provided motion signal. Moreover, since this directly influenced motion signal is used by the determination device for determining the physiological parameter, the physiological parameter can be determined with an increased sensitivity.

This is in contrast to the measurement described in the above mentioned articles by P. Sharma et al. and J. Lin et al., in which the measured signal is related to electrical changes close to the skin surface only, i.e. it is not related to mechanical motion of a structure within the subject. For instance, if the structure is the heart, the RF radiation does not penetrate into the heart due to the high RF transmit frequency, which limits the sensitivity. Furthermore, the measurements described in these articles do not allow to determine many different cardiac parameters. For instance, it is not possible to quantify stroke volume of the heart with the measurements disclosed in these articles.

Preferentially, the measurement device is configured to be worn by the subject. However, it is also possible that the measurement device is not configured to be worn by the subject. For instance, it can be a handheld device to be held in front of the subject like on the chest, in order to determine the physiological parameter. The measurement device can also be configured to be arranged on wall or to be arranged on a rack, stage or the like, wherein the subject can be arranged in front of the measurement device for determining the physiological parameter.

The RF instrument can be configured to directly provide the received RF signal as the motion signal. However, the RF instrument can also be configured to process the received RF signal and to provide the processed RF signal as the motion signal. Moreover, the RF instrument preferentially is configured to separate the received RF signal from the transmitted RF signal, i.e. from the RF power transmitted into the RF antenna module, if required. In a preferred embodiment the RF instrument is a vector network analyzer. Moreover, in an embodiment the RF instrument is configured to use a bi-directional coupler or to carry out the transmitting and receiving procedure after each other, in order to separate the transmitted RF signal from the received RF signal.

Preferentially, the processor is configured to determine at least one of a heart-related physiological parameter and a lung-related physiological parameter. For instance, the processor can be configured to determine at least one of the heart rate and the stroke volume as the heart-related physiological parameter. Moreover, the processor can be configured to determine at least one of the respiratory rate and the tidal volume as the lung-related physiological parameter.

The measurement device preferentially comprises a transmitter configured to transmit the measured signal, particularly the motion signal, to the determination device. In particular, the measurement device and the determination device are separate devices which are connected via a wireless data connection like Bluetooth.

In a preferred embodiment, the model providing module is a storage like a storage of a mobile device or of a personal computer, in which the model is stored and from which the model can be obtained, and the processor can be a processor of the mobile device or of the personal computer, respectively. The mobile device can be, for instance, a smartphone, a tablet computer or a laptop.

The model providing module can be a storage, as mentioned above, in which the model is stored and from which the model can be obtained, but the model providing module can also be a receiving unit configured to receive the model from another device like another storage. It is also possible that the model providing module is configured to generate the model or adapt a present model by training or calibration and to provide the created or adapted model to the processor.

In an embodiment the RF instrument, which in a preferred embodiment is a vector network analyzer, is configured to provide as the motion signal a complex signal. In particular, the RF instrument is configured to provide at least one of a) a complex reflection coefficient and b) a complex coupling coefficient as the motion signal. In a preferred embodiment, the processor is configured to identify a first subsignal of the complex signal having a distinct phase shift of, for example, 90 degrees with respect to a second subsignal of the complex signal and to determine the physiological parameter based on at least one of the identified subsignals, for example based on the first subsignal. Thus, the processor can be configured to process the motion signal such that a processed motion signal is obtained, i.e. for instance the identified first subsignal, and to determine the physiological parameter based on this processed motion signal.

It has been found that the complex signal can comprise contributions from at least two subsignals having a distinct phase shift relative to each other of, for instance, 90 degrees. In particular, one of these subsignals can be caused by cardiac motion and the other of these subsignals can be caused by respiratory motion. Thus, by identifying the first subsignal and using the identified first subsignal for determining a heart-related physiological parameter, the determination of the heart-related physiological parameter can be less influenced by respiratory motion, thereby allowing for an increased accuracy of determining the heart-related physiological parameter. Preferentially, the distinct phase is a predetermined phase, wherein the predetermined phase can be predetermined by, for example, a calibration procedure.

In a preferred embodiment the RF instrument and the RF antenna module are configured to be operated within an operating frequency from 30 to 300 MHz and further preferred in a frequency range from 100 to 150 MHz. It has been found that, if the operating frequency is within this frequency range, the distinct phase shift is relatively accurately 90 degrees, which further increases the quality of separating the heart-related subsignal from the other subsignal. This allows for a further increased accuracy of determining particularly a heart-related physiological parameter based on the heart-related subsignal.

The processor can be configured to determine the heart-related subsignal, i.e. the processed motion signal to be used for determining the heart-related subsignal, such that it has a maximum absolute or relative magnitude in a predefined expected frequency range in which the heart frequency is expected. This expected frequency range can be, for instance, 0.7 Hz to 1.5 Hz. In particular, the determination device can be configured to perform a phase rotation on the complex motion signal, which has been received from the measurement device and which is to be processed for generating the processed motion signal, such that the heart-related subsignal, i.e. the processed motion signal, is aligned with the real axis of a corresponding complex coordinate system. This can be performed by rotating the received complex motion signal in the complex coordinate system until the real part of the rotated complex motion signal has reached a maximum absolute or relative magnitude in the predefined expected frequency range in which the heart frequency is to be expected. For determining the magnitude of the subsignal in the predefined expected frequency range, the subsignal is preferentially transformed into the frequency domain by using, for instance, a Fourier transform. Thus, in an embodiment, a phase rotation is performed on the complex motion signal until the magnitude of the real part of the rotated complex motion signal has a maximum in the predefined expected frequency range, wherein, especially if the different subsignals related to different kinds of motion are separated by 90 degrees, the resulting subsignal, i.e. the resulting real part of the rotated complex total motion signal, is substantially only heart-related. The processor then can use this resulting subsignal, i.e. the processed motion signal in which respiratory influences have been reduced or even eliminated, for determining the heart-related physiological parameter with high accuracy.

The absolute magnitude of the subsignal in the predefined expected frequency range of, for instance, 0.7 Hz to 1.5 Hz can be, for instance, the maximum value within this frequency range or it can be the output of a function which has, as inputs, one or several magnitude values in the predefined frequency range. For example, the absolute magnitude of the subsignal in the predefined expected frequency range, which should be maximized, can be the average of the magnitude values in the predefined expected frequency range.

This absolute magnitude of the subsignal in the predefined expected frequency range can be directly used for finding the heart-related subsignal or it can be related to one or several magnitudes outside of the predefined expected frequency range for forming the relative magnitude of the predefined expected frequency range. For instance, the absolute magnitude of the subsignal in the predefined expected frequency range can be compared with the magnitude of the subsignal in another predefined unwanted frequency range that should be suppressed like a frequency range from 0.15 Hz to 0.25 Hz, if respiratory motion should be suppressed. For this comparison, an unwanted magnitude can be determined based on one or several magnitudes within the unwanted frequency range. For example, the unwanted magnitude can be the maximum magnitude within the unwanted frequency range or it can be the average of the magnitudes within the unwanted frequency range.

It is also possible that the absolute magnitude of the subsignal in the predefined expected frequency range is compared with another unwanted magnitude like a background magnitude. The background magnitude is the background with respect to the predefined expected frequency range. Thus, the background magnitude can be determined as the average of the magnitudes of the subsignal outside of the predefined expected frequency range.

The comparison for providing the relative magnitude of the expected frequency range can be carried out by subtracting the absolute magnitude of the subsignal in the predefined expected frequency range by a) the magnitude of the subsignal in the predefined unwanted frequency range, b) the background magnitude or c) a combination of the unwanted magnitude and the background magnitude. Also another comparison measure can be used like a division. Hence, the comparison for providing the relative magnitude of the expected frequency range can be carried out by dividing the absolute magnitude of the subsignal in the predefined expected frequency range by a) the magnitude of the subsignal in the predefined unwanted frequency range, b) the background magnitude or c) a combination of the unwanted magnitude and the background magnitude.

The processor can be configured to determine the subsignal, i.e. the processed motion signal, such that the comparison measure yields a maximum value. In particular, the total complex signal, i.e. the initially received motion signal, can be rotated in the complex coordinate system, i.e. the phase can be rotated, until the comparison has reached its maximum for the real part of the total complex signal, wherein this real part is the processed motion signal that is used by the processor for determining the physiological parameter.

In a preferred embodiment the processor is configured to apply a blind source separation technique, in order to generate a processed motion signal, and to use the processed motion signal and the provided model to determine the physiological parameter. Moreover, in an embodiment the model providing module is configured to provide as the model at least one of a linear regression model, a polynomial regression model and a Gaussian process regression model.

Thus, in an embodiment the processor is configured to apply a blind source separation technique such as independent component analysis (ICA) or principal component analysis (PCA) to the motion signal that has been received from the measurement device, in order to generate a processed motion signal, and to use the processed motion signal, which can also be regarded as being a subsignal of the initial motion signal, to determine the physiological parameter. It has been found that processing the motion signal by using second order blind identification (SOBI) yields even more accurate physiological parameters and therefore is preferred. Moreover, the processor can be configured to apply a frequency filtering to the motion signal and to use the resulting processed motion signal to determine the physiological parameter. The frequency filtering can be, for instance, a band-pass filtering, a low-pass filtering, a high-pass filtering or a Kalman filtering. This further processing of the measured signal finally allows for a further increased accuracy of determining the physiological parameter.

In particular, if the motion signal, which is received from the measurement device, is a complex signal, it in fact comprises two subsignals, for example, magnitude and phase or real part and imaginary part. The blind source separation technique like a principal component analysis (PCA) can be applied to these two subsignals. In particular, a vector can be defined with two vector elements, wherein the first vector element comprises one of the magnitude and phase and the second vector element comprises the other of the magnitude and phase. It is also possible that the first vector element comprises one of the real part and the imaginary part and the second vector element comprises the other of the real part and the imaginary part. The blind source separation technique can be applied to this vector, thereby generating a new vector, wherein the first vector element of the new vector is a first subsignal and the other vector element of the new vector is a second subsignal. These two subsignals are independent, uncorrelated or orthogonal with respect to each other due to the blind source separation technique. In order to determine which subsignal should be used for determining which physiological parameter, a frequency analysis can be performed on the two subsignals. In particular, the amplitude of the respective subsignal in a predefined expected frequency range, which is expected to be indicative for the respective physiological parameter to be determined, can be compared with the amplitude of the respective subsignal in one or several other frequency ranges, in particular, in all other frequency ranges. For instance, a Fourier transform can be carried out, in order to transform the respective subsignal into the frequency domain, wherein the value of the respective frequency spectrum in the expected frequency range can be compared with the value of the respective frequency spectrum outside of the expected frequency range, in particular, it can be compared with the value of the respective frequency spectrum in another unwanted frequency range which is indicative of unwanted motion to be suppressed like respiratory motion, if a heart-related parameter should be determined. The value of the respective frequency spectrum in the expected frequency range can also be compared with the average value of the respective background signal being defined as the average of the value over the whole frequency range excluding the expected frequency range. The comparison can be carried out by division, subtraction or another comparison measure. The subsignal, for which the value in the expected frequency range relative to the value in another unwanted frequency range being indicative of unwanted motion to be suppressed or relative to the background signal is largest, is selected to be the subsignal, i.e. the processed motion signal, which should be used by the processor for determining the physiological parameter. If the physiological parameter is a cardiac parameter, the expected frequency range can be, for instance, 0.7 Hz to 1.5 Hz and the unwanted frequency range can be, for instance, 0.15 Hz to 0.25 Hz. If the physiological parameter is a respiration-related parameter, the expected frequency range can be, for instance, 0.15 to 0.25 Hz and the unwanted frequency range can be, for instance, 0.7 Hz to 1.5 Hz.

It is also possible that the processor applies at least one of the blind source separation and the frequency filtering to the motion signal, which has been received from the RF instrument, for generating the processed motion signal to be used for determining the physiological parameter.

Preferentially, the model providing module is configured to provide a linear model as the model. It has been found that already a linear model can lead to a determination of a physiological parameter like a stroke volume or a heart rate with an increased accuracy such that relatively low computational efforts are required for, for instance, training the model and utilizing the model.

Furthermore, preferentially the RF instrument, particularly the vector network analyzer, and the RF antenna module are configured to be operated in a frequency range from 30 to 1000 MHz, further preferred in a frequency range from 300 to 800 MHZ. Moreover, in a preferred embodiment the RF instrument and the RF antenna module are configured to be operated in a frequency range from 30 to 300 MHZ. If an operating frequency within this frequency range is used, the RF radiation causes power deposition throughout the whole body including the structure like the heart. Thus, this frequency range can be used to generate a signal related to the mechanical motion of the structure.

In an embodiment the RF instrument and the RF antenna are configured to be operated with an operating frequency of 64, 128 or 300 MHz. These operating frequencies are equal to the Larmor frequency of a magnetic resonance imaging (MRI) system at 1.5 T, 3 T and 7 T, respectively. Thus, as it will be explained further below, if one of these operating frequencies is used, the model can be trained very effectively by using an MRI system having a main magnetic field strength of 1.5 T, 3.0 T or 7.0 T, respectively.

As mentioned above, in an embodiment the RF instrument and the RF antenna are configured to be operated in a frequency range from 300 to 800 MHZ. An operating frequency within this frequency range can be advantageous, if a single smaller structure like a smaller organ such as the heart should be monitored, i.e., for instance, if a motion signal related to a mechanical movement of the heart should be provided, because in this case the RF radiation does not cause power deposition throughout the whole body, but only in a region closer to the RF antenna module like a heart region if the RF antenna module is arranged on the subject's breast region.

In an embodiment, the measurement device is configured to measure different motion signals for different frequencies, wherein the determination device is configured to determine the physiological parameter based on the motion signals measured for the different frequencies. The different motion signals for the different frequencies can be acquired by one RF antenna or by several RF antennas. In an embodiment, the different motion signals can be regarded as being a motion signal which depends on the frequency. In particular, in an embodiment the RF instrument is configured to transmit RF power into the RF antenna module with different frequencies, in order to provide the motion signal for the different frequencies, wherein the determination device is configured to determine the physiological parameter based on the motion signal provided for the different frequencies. Thus, measurements can be performed, wherein the operating frequency is changing in time. In particular, a frequency sweep can be performed. By measuring at multiple frequencies, both global and local motion effects can be distinguished, which can improve the accuracy of determining the physiological parameter. In an embodiment, different motion signals measured at different frequencies can be used to measure different physiological parameters, for example heart rate, stroke volume and tidal volume are measured at different frequencies. In another embodiment, motion signals at multiple frequencies can be combined, for example by averaging or a blind source separation technique like ICA, PCA or most preferentially SOBI, in order to determine a processed motion signal that can be used to determine the physiological parameter with a further improved accuracy.

In an embodiment, the different frequencies cover a range from 30 MHz to 1300 MHz. For instance, the different frequencies can be 34 MHZ, 67 MHZ, 100 MHZ, 134 MHZ, 167 MHZ, 200 MHZ, 234 MHZ, 267 MHZ, 300 MHZ, 334 MHz, 367 MHZ, 400 MHZ, 434 MHz, 467 MHz, 500 MHz, 534 MHz, 567 MHZ, 600 MHZ, 633 MHZ, 667 MHZ, 700 MHZ, 733 MHZ, 767 MHZ, 800 MHZ, 833 MHZ, 867 MHZ, 900 MHZ, 933 MHz, 967 MHZ, 1000 MHz, 1033 MHz, 1067 MHZ, 1100 MHZ, 1133 MHZ, 1167 MHZ, 1200 MHZ, 1233 MHZ, 1267 MHz and 1300 MHz. Thus, the frequencies, at which the motion signal is measured, can be equidistantly distributed over the range of 30 MHz to 1300 MHz. However, the different frequencies can also cover a smaller range like 30 MHz to 1000 MHZ, 30 MHz to 300 MHz or 100 MHZ to 150 MHz. Also if the frequency range is smaller than from 30 MHz to 1300 MHZ, the different operation frequencies, at which the RF signal is measured, are preferentially equidistantly distributed over the frequency range.

The measurement device can be configured to measure different motion signals for different frequencies, wherein the determination device can be configured to combine the motion signals, which have been measured for the different frequencies, and to determine the physiological parameter based on the combined motion signals. Thus, the motions signals, which are received from the RF instrument, are combined and thereby processed for determining a processed motion signal that can be used by the processor for determining a physiological parameter. The combination of the motion signals can be a linear combination. The linear combination can be determined by a blind source separation technique like a PCA or ICA. In particular, the processor can be configured to determine the physiological parameter based on the first principal component being, in this example, the processed motion signal. For instance, the model can provide a relation between the first principal component, i.e. the processed motion signal, and the physiological parameter, wherein the processor can be configured to determine the physiological parameter based on the first principal component and the relation. The relation and hence the model can be predetermined by calibration. It can be a linear relation. In an embodiment, the first principal component can be used for determining a heart-related physiological parameter like the stroke volume. The second principal component, which is a further processed motion signal, can be used, for instance, for determining a lung-related parameter.

In an embodiment, the motion signals obtained from the measurement device are complex and have been measured at different frequencies simultaneously with a gold standard measurement of the physiological property in a training phase. In particular, the gold standard measurement can be a measurement of the stroke volume by using transthoracic echo or magnetic resonance imaging (MRI). Since each received motion signal, which has been measured at the respective frequency, is complex, each motion signal in fact is formed by two subsignals like a phase subsignal and a magnitude subsignal or a real part subsignal and an imaginary part subsignal. The different subsignals, which have been measured at different frequencies, can be combined by using a blind source separation like PCA. Depending on the used blind source separation technique, the number of resulting separated subsignals can vary between two and the total number of initial subsignals. The new subsignals, i.e. the processed motion signals obtained by applying the blind source separation technique, are compared with the gold standard physiological parameter, wherein among the new subsignals the subsignal is selected, which correlates best with the gold standard physiological parameter. This can be carried out by using a comparison measure like a calculation of root-mean-square error or a calculation of a correlation, wherein the new subsignal having the lowest root-mean-square error or the highest correlation with the gold standard physiological parameter can be selected to be used in future physiological parameter determination procedures. Thus, this part of the training phase determines which new subsignal, for instance, in case of SOBI, which SOBI component, should be used for determining the physiological parameter. In a preferred embodiment, one of the first and second SOBI components, particularly the first SOBI component, is used for determining a heart-related physiological parameter or a lung-related physiological parameter.

The model which should provide the relation between the selected new subsignal, i.e. the processed motion signal, and the physiological parameter can also be determined in the training phase, wherein a linear regression model, a polynomial regression model or most preferentially a Gaussian process regression model can be used. In particular, the corresponding model includes one or several parameters which are modified such that, if the model is used together with the selected subsignal for determining the physiological parameter, this determined physiological parameter corresponds as good as possible to the gold standard physiological parameter. This training and also the other trainings described in this patent application can be done on a subject specific basis or on a group basis. After this training phase has been completed, the determination device can use the training result for determining the physiological parameter in future determinations. In particular, the same kind of combining the initially received motion signals, the same resulting new subsignal, i.e. the same processed motion signal, and the same adapted model is used by the processor for determining the physiological parameter based on future RF measurements.

It has been found that a particularly accurate physiological parameter can be determined, if, as the processed motion signal, a SOBI component is used and as the model a Gaussian process regression model is used. In particular, one of the first and second SOBI components, particularly the first SOBI component, can be input into the Gaussian regression model for determining a heart-related physiological parameter or a lung-related physiological parameter.

In another embodiment, the subsignals of the acquired complex motion signals are directly used, i.e. they are not processed by using a blind source separation technique, for the comparison with the gold standard physiological parameter. For instance, the processor can be configured to determine which received subsignal of the received complex motion signals has the best correlation with the measured gold standard physiological parameter, wherein this best correlation subsignal can be used by the processor together with a corresponding model for determining the physiological parameter in future measurements. The correlation could be determined, for example, by regression analysis, Bland-Altman analysis, calculation of a root-mean-square error between the respective subsignal and the gold standard physiological parameter which, because of being measured over time, is also a signal, or by using another correlation measure. After this training, the processor can use the same selected subsignal in an actual measurement for determining the physiological parameter. Also in this embodiment, a corresponding model can be trained to provide, as an output, the physiological parameter, if, as an input, the selected type of subsignal, i.e. the selected motion signal, is provided. In a further embodiment, during a training phase, a subsignal of the received motion signals can be selected by comparing the absolute magnitude of the respective subsignal in a predefined expected frequency range or the relative magnitude of the respective subsignal in the predefined expected frequency range of the different subsignals with respect to each other, as described above. The subsignal, which has the highest absolute magnitude or relative magnitude within the predefined expected frequency range then can be selected, wherein this selected processed motion signal can be used together with the gold standard physiological parameter for training the model, which in future measurements can be used by the processor for determining the actual physiological parameter.

In order to measure the different motion signals for the different frequencies, the measurement device, in particular the RF instrument and the RF antenna, can be configured to be operated at multiple frequencies in a frequency sweep. The sequentially obtained signals at different frequencies have different penetration depths and result in motion signals that can be acquired with the same sensor, i.e. with the same RF instrument and the same RF antenna. By combining the signals acquired at different frequencies, the accuracy of the measurement and sensitivity to breathing and bulk motion related artifacts can be minimized. Thus, for instance, the heart-related physiological parameter can be determined even more accurately.

The RF antenna module is matched to the characteristic impedance of the RF instrument which generally is 50Ω. Moreover, preferentially the reflection coefficient of the RF antenna module is lower than −1 dB and further preferred lower than −3 dB for the operation frequency. Furthermore, if the measurement device should be used with several operation frequencies, it is desirable that the RF antenna module has a large bandwidth, which can be defined as the frequency span within which the reflection coefficient is lower than −1 dB and further preferred lower than −3 dB. In a preferred embodiment, the RF antenna module is constructed such that it has multiple resonance frequencies within a range spanned by the bandwidth. Preferred corresponding RF antenna modules having multiple resonance frequencies are described further below.

Preferentially, the RF antenna module has a width and a length which are each smaller than 20 cm. In particular, the dimensions of the RF antenna module are such that it can be arranged within a virtual sphere having a diameter of 20 cm. Thus, preferentially the RF antenna module is not too large, in order to allow the RF antenna module to be relatively easily integrated into the measurement device, which might be configured to be worn. In particular, the RF antenna module can be integrated into a holder of the measurement device for holding the RF antenna module. In a preferred embodiment, the RF antenna module has a width and a length which are each within a range of 5 cm to 20 cm and further preferred within a range from 10 cm to 15 cm. Thus, in a preferred embodiment, the RF antenna module is configured such that it can be arranged within a virtual sphere having a diameter being equal to or smaller than 20 cm, but it cannot be arranged within a virtual sphere having a diameter being equal 5 cm, and, in a further preferred embodiment, the RF antenna module is configured such that it can be arranged within a virtual sphere having a diameter being equal to or smaller than 15 cm, but it cannot be arranged within a virtual sphere having a diameter being equal 10 cm. An RF antenna module with these dimensions is optimized for providing a motion signal being indicative of cardiac motion.

Moreover, preferentially the RF antenna module comprises one or more dipole antennas or loop coils as the one or more RF antennas. Dipole antennas have the benefit that they have a localized sensitivity and their electric field radiates deeply into tissue, which can allow for a further improved accuracy of determining the physiological parameter.

In an embodiment, the one or more RF antennas include at least one of a dipole antenna with a gap and a loop coil with a gap in which a capacitor is arranged, wherein it has been found that by using such an RF antenna with a gap the accuracy of determining the physiological parameter can be even further increased.

Preferentially, the dipole antenna comprises a straight conductive element like a conductive wire or conductive strip, which has a gap in its center. Thus, due to the gap, the conductive element comprises two separate conductive subelements which might be named “legs”. At the location of the gap, a matching circuit and an excitation source are placed, which connect the two separate conductive subelements. The matching circuit and the excitation source can be a well-known matching circuit and a well-known excitation source. In an embodiment, the length of the conductive element is half of the RF wavelength which corresponds to the operating frequency. The length of the conductive element can also be shorter or longer depending on the elements of the matching circuit. Also for amending the length of the conductive element a known matching circuit can be used. The matching circuit can comprise inductors and/or capacitors that tune the dipole antenna to the desired operating frequency.

In an embodiment, the two separate conductive subelements, i.e. the legs, have a T-like shape at their ends that face away from each other, i.e. at their outer ends. The T-like ends can increase the bandwidth and increase the sensitivity perpendicular to the longitudinal axis of the straight conductive element. It has been found by the inventors that a total antenna length of 180 mm, i.e. a total length of the straight conductive element or, in other words, a length from the end of one of the T-shapes to the end of the other of the T-shapes of 180 mm, a smaller width of the respective T-like shape of 30 mm and a larger width of the respective T-like shape of 50 mm provides an optimized tradeoff between sensitivity to cardiac motion and bandwidth. In a preferred embodiment, the dipole antenna has these geometric dimensions or at least dimensions that are within 10% of the previously provided optimized values. It should be noted that the length direction of the dipole antenna is defined by the longitudinal axis of the straight conductive element and the width direction is defined as being perpendicular to this longitudinal axis.

Furthermore, an additional matching circuit can be used to match the dipole antenna to a cable like a coaxial cable for a maximized transmission efficiency. This cable is preferentially the cable to the RF instrument being preferentially a vector network analyzer. This additional matching circuit can be, for instance, a lattice balun as disclosed, for example, in the article “Lumped and Distributed Lattice-type LC-Baluns” by W. Bakalski et al., 2002 IEEE MTT-S International Microwave Symposium Digest, DOI: 10.1109/MWSYM.2002.1011595, which is herewith incorporated by reference.

Preferentially, the dipole antenna has higher order resonance frequencies at frequencies at which the length is equal to a positive integer multiple of the respective wavelength. The corresponding bandwidth, which might be measured as full width at half maximum of the reflection, can be increased, by increasing the width of the conductive element. Thus, the width is preferentially increased, in order to make the dipole antenna better usable at a wide range of operating frequencies.

Loop coils have the benefits that they can be made very small, even for low transmit frequencies, which allows for an improved integration of the RF antenna module into the measurement device, particularly into a holder of the measurement device.

Preferentially, the loop coil comprises a circular, rectangular or octagonal conductive element like a correspondingly shaped wire or conductive strip, which has a gap in which a matching circuit and an excitation source are placed. In an embodiment, the loop coil has multiple gaps in its conductive element, wherein a respective capacitor is placed in the respective gap. The RF instrument can be configured to operate the loop coil in a loop mode. The inductance of the conductive element in combination with the multiple capacitors makes the loop coil resonant at a respective frequency ω=1/\sqrt{LC}, wherein L is the total inductance of the conductive element, which might be circular, and C is the total capacitance of the multiple capacitors. The loop coil is operated in the loop mode by using relatively low operation frequencies for which the resonant condition is fulfilled. Correspondingly, in the loop mode the current runs substantially uniformly along the whole length of the conductive element which, as mentioned above, preferentially is circular.

The RF instrument can also be configured to operate the loop coil in a dipole mode. In the dipole mode, the loop coil acts like a dipole antenna. In the dipole mode the RF instrument uses an operation frequency being so large that the current on the conductor decreases to 0 before it reaches the capacitors due to increased resistive losses at the higher frequency. Correspondingly, in the dipole mode, only the subconductive elements connected to the RF instrument comprise current and these conductive subelements, in which the current runs, act like a dipole antenna. By operating the loop coil in the loop mode and in the dipole mode, the loop coil can be resonant at multiple frequencies, thereby improving the sensitivity over a large frequency range, wherein this in turn can improve the accuracy of determining the physiological parameter.

In a preferred embodiment, the diameter of the conductive element of the loop coil, for instance, the diameter of a circular conductive element of the loop coil, is within a range from 70 mm to 110 mm. It has been found by the inventors that a loop coil with a diameter within this range allows to provide a motion signal that is related to a mechanical movement of the heart with a further increased accuracy. Moreover, in a preferred embodiment the loop coil is tuned to 433 MHz as a first resonance frequency and to 920 MHz as a second order resonance frequency. These frequencies correspond to radio bands that are reserved for industry, medical and scientific (ISM) purposes.

Preferentially, the loop coil is connected to the RF instrument by using a coaxial cable, wherein a matching inductor or alternatively a capacitor and a lattice balun can be used to match the impedance of the loop coil to the characteristic impedance of the coaxial cable.

In an embodiment, the RF antenna module comprises at least two RF antennas, wherein these at least two RF antennas are a dipole antenna and a loop coil. In this case, preferentially the connection of the RF antenna module to the RF instrument is such that RF power is transmitted from the RF instrument into one of the dipole antenna and the loop coil and the RF signal received by the other of the dipole antenna and the loop coil is measured and used for providing the motion signal that is related to the mechanical movement of the structure within the subject. Thus, the motion signal is provided based on the RF signal received by the other of the dipole antenna and the loop coil. By using one of the dipole antenna and the loop coil for transmission and the other of the dipole antenna and the loop coil for reception, the amount of inherent coupling between transmission and reception can be very low due to geometrical decoupling. The RF signal received by using the other of the dipole antenna and the loop coil is affected only by the subject and insensitive to artifacts from the surrounding.

In an embodiment, a) the RF antenna module comprises one RF antenna and the measurement device is configured such that, if the measurement device is worn by an adult, a center point of the RF antenna is positioned left to the sternum, or b) the RF antenna module comprises at least two RF antennas, in particularly only two RF antennas, and the measurement device is configured such that, if the measurement device is worn by an adult, a center point of one of the at least two RF antennas is positioned left to the sternum and a center point of the other of the at least two RF antennas is positioned right to the sternum. In a preferred embodiment in which the RF antenna module comprises a single RF antenna only, it is positioned with a shift within the range of 2 cm to 4 cm, further preferred with a shift of 3 cm, to the left of the sternum. This allows to position the RF antenna directly above the heart, thereby allowing to determine a heart-related physiological parameter very accurately like the heart rate or the stroke volume. In a further preferred embodiment in which the RF antenna module comprises at least two RF antennas, particularly only two RF antennas, one of the RF antennas is positioned with a shift within the range of 2 cm to 4 cm, further preferred with a shift of 3 cm, to the left of the sternum and the other of the RF antennas is positioned with a shift within the range of 2 cm to 4 cm, further preferred with a shift of 3 cm, to the right of the sternum. If such a configuration is used, one of the RF antennas is located directly above the heart, wherein the signal generated by using this RF antenna can be used for very accurately determining a heart-related physiological parameter. The other RF antenna has a relatively large distance to the heart and is located directly above the lungs such that the signal generated by using the other RF antenna can be indicative of the influence of the movement of the lungs on the signal which has been generated by the RF antenna located directly above the heart. The signal generated by the other RF antenna can hence be used for correcting the signal generated by using the RF antenna directly above the heart such that the influence of the motion of the lungs can be removed or at least reduced in the signal measured by using the RF antenna above the heart. The signal correction can be performed by using digital signal processing techniques to separate the heart and lung motion from the set of received signals, for example PCA, ICA, or other blind source separation techniques. This can lead to a further increased accuracy of determining a heart-related physiological parameter.

Moreover, in a preferred embodiment in which the RF antenna module comprises two RF antennas only, the RF instrument and the two RF antennas are configured such that an inter-element coupling between the two RF antennas is below a predefined value, wherein this predefined value might be, for instance, −12 dB. In particular, the distance between the two RF antennas is such that the inter-element coupling between the two RF antennas is below −12 dB. This distance can be, for instance, within a range of 4 to 8 cm and preferentially is 6 cm, wherein the distance refers to the distance between the center positions of the two RF antennas. This can lead to a further increased accuracy of determining the physiological parameter.

In an embodiment the RF antenna module includes several RF antennas having different transmit phases defining a sensitivity profile of the RF antenna module, wherein the RF antenna module is configured such that the sensitivity profile has its largest sensitivity at the location of the structure which preferentially is an organ. Also this allows for a further increased accuracy of determining the physiological parameter.

In an embodiment the RF antenna module comprises several RF antennas which are arranged in a belt-like configuration. In particular, the belt-like configuration can be such that the RF antennas are arranged around a torso of the subject. In an embodiment, the number of RF antennas in the belt-like configuration is within a range from 3 to 32 RF antennas. This configuration can allow to improve the sensitivity of the measurement device to the physiological parameter. In particular, if several RF antennas are arranged in a belt-like configuration around the structure, a sensitivity profile of the RF antenna module comprising the several RF antennas can be adapted such that a very focused maximum sensitivity is at the location of the structure, thereby allowing for a further increased accuracy of determining the physiological parameter. For example, the phase of the signals transmitted to the RF antennas can be modified in such a way that the sensitivity to the physiological parameter is as high as possible. Optimal transmit phases can be determined by comparing measurements that use different transmit phases or by performing an electromagnetic simulation of the full measurement system. The electromagnetic simulations can be performed with different transmit phases to determine optimal measurement settings. The RF antennas of the belt-like configuration receive a multitude of signals. To determine the physiological parameter from these multiple signals, digital signal processing techniques such as PCA, independent component analysis or other blind source separation techniques can be used to separate different contributions to the signals and isolate measurements of physiological parameters.

In an embodiment the RF antennas are arranged in multiple rows. For instance, they can be arranged in up to eight rows, wherein preferentially each row comprises a number of RF antennas within a range from 3 to 32. Thus, in an embodiment the RF antenna module comprises 256 RF antennas. Preferentially, each row of RF antennas has a limited field of view in the feet-head direction, wherein this field of view might be defined by the respective region within which the respective RF antenna has a sensitivity being above 50% of its maximum sensitivity. This field of view can have a size in the feet-head direction of, for instance, 5 cm. This allows to locally receive RF signals and hence to provide a local motion signal, wherein this local motion signal can be used by the determination device for determining a spatial distribution of one or several physiological parameters.

In an embodiment, the local RF signals are provided to the determination device as local motion signals, which are processed by the processor of the determination device. In particular, the processor can be configured to combine the received motion signals of the multiple RF antennas, in order to determine a processed motion signal, wherein the model providing unit can then be configured to provide a model that provides, as an output, a physiological parameter if, as an input, the processed motion signal is provided and wherein the processor can be configured to determine the physiological parameter based on this provided model and the processed motion signal. The model can be, for instance, a linear model or most preferentially a Gaussian process regression model. The received RF signals, i.e. the received motion signals, can be combined to a processed motion signal by using blind source separation like PCA, ICA or most preferentially SOBI. For instance, SOBI can be carried out and a SOBI component can be used as the processed motion signal. The model can be trained in a training phase, wherein in the training phase ground truth measurements of the physiological parameter are compared with the physiological parameter that is determined by the processor, wherein the model, in particular parameters of the model, are modified until a deviation between the ground truth physiological parameter and the physiological parameter determined by the processor by using the modified model is minimized. The ground truth measurement can be, for instance, an MRI measurement yielding the left ventricular volume if the left ventricular volume is the physiological parameter to be determined.

In a further embodiment one or several RF antennas can be arranged at predefined anatomical structures which are close to a structure within the subject of which a motion signal should be provided. For instance, for providing a motion signal that is related to the left ventricular movement or the right ventricular movement, which might be used for determining the left ventricular volume or the right ventricular volume, respectively, one or several RF antennas can be placed close to the left ventricle and one or several RF antennas can be placed close to the right ventricle, respectively. Moreover, for providing a motion signal that is related to a mechanical movement of the aorta, one or several RF antenna modules can be placed close to the aorta.

In an embodiment, the RF antenna module comprises a loop coil having a diameter within a range from 70 mm to 170 mm and preferentially within a range from 70 mm to 110 mm. Moreover, in an embodiment the loop coil comprises a conductive element with several gaps in which a respective capacitor is placed, as explained above, wherein the capacitor in the gap close to the connection to the RF instrument can be placed at the midsternal line sternum, in particular at the height of the fourth intercostal space. In particular, the loop coil can be arranged in a wearable holder, wherein the wearable holder and the loop coil can be arranged such that the capacitor close to the connection to the RF instrument is placed at the midsternal line, preferentially at the height of the fourth inter-coastal space, if the wearable holder is worn by the subject.

The measurement device can include, as mentioned above, a wearable holder comprising at least the one or more RF antennas of the RF antenna module. Moreover, preferentially, the one or more RF antennas of the RF antenna module are flexible. Due to the flexibility of the one or more RF antennas, they can be better integrated into the wearable holder and wearing the measurement device is more convenient for the subject. The wearable holder can comprise a visible marker assigned to an anatomical feature of the subject, wherein the wearable holder can be configured to be worn such that the marker is arranged at a position on the subject at which the assigned anatomical feature is located. In a preferred embodiment, the anatomical feature is the sternum at the height of the nipples. This ensures that the measurement device is worn correctly by the subject such that the one or more RF antennas are positioned at locations on the subject, which allow for the accurate determination of the physiological parameter.

In an embodiment the RF antenna module includes at least a first RF antenna and a second RF antenna, wherein the RF instrument and the RF antenna module are configured to provide a first motion signal of the first RF antenna that is related to a mechanical movement of a first structure within the subject and to provide a second motion signal of the second RF antenna that is related to a mechanical movement of a second structure within the subject, wherein the processor is configured to process the motion signals such that in a first processed motion signal contributions of the movement of the second structure are removed, wherein the processor is configured to determine the physiological parameter based on the first processed motion signal. In particular, the RF instrument, which preferentially is a vector network analyzer, is configured to determine a coupling between the first RF antenna and the second RF antenna. The coupling between the first RF antenna and second RF antenna can be determined by measuring the signal scattered from the first antenna to the second antenna. The RF instrument preferentially has two connecting ports, where one port is able to transmit signal into the first RF antenna and the other port receives signal from the second RF antenna. The coupling signal can be used to remove contributions of the movement of the second structure to the first motion signal from the first motion signal based on the determined coupling and to determine the physiological parameter based on the first motion signal, i.e. based on the resulting first processed motion signal.

In particular, the processor is configured to apply digital signal processing techniques to the first motion signal and the second motion signal like a blind source separation technique, in order to remove contributions of the movement of the second structure to the first motion signal from the first motion signal and optionally also to remove contributions of the movement of the first structure to the second motion signal from the second motion signal. For instance, a PCA, an ICA, a SOBI or another blind source separation method can be applied to the first and second motion signals.

In order to measure the coupling between the first RF antenna and the second RF antenna, the RF instrument, which preferentially is a vector network analyzer, can provide a harmonic signal to the first RF antenna that can be connected to a port of the RF instrument like to “port 1” of the RF instrument. Moreover, the second RF antenna can also be connected to the RF instrument, for instance, it can be connected to a further port of the RF instrument, which might be named “port 2”. The RF instrument then can measure the magnitude and phase of the power received via the second RF antenna, while the RF instrument provides a harmonic signal to the first RF antenna. In a preferred embodiment, the signal received by the second RF antenna, while another signal is provided to the first RF antenna, is regarded as being the coupling between the two RF antennas. In particular, the RF instrument can be configured to measure a first RF signal at the first RF antenna, which can be regarded as being a reflection of the first RF antenna and which might also be regarded as being a first motion signal, and a second RF signal at the second RF antenna, which can be regarded as being the coupling. Moreover, preferentially these signals are complex, such that in fact four distinct subsignals are measured, i.e. for each of the two complex signals a respective amplitude and magnitude signal or a respective imaginary and real signal are provided as sub-RF signals. These four signals can be combined by the processor, in order to determine a first processed motion signal by using, for instance, a blind source separation technique like PCA, ICA or most preferentially SOBI.

In an embodiment, the first RF antenna is placed close to the heart and the second RF antenna is placed on the torso far away from the heart such that the amplitude and the magnitude of the first RF antenna signal, i.e. the first motion signal being a reflection signal, is mostly affected by cardiac motion and less affected by respiratory motion and such that the magnitude and phase of the second RF antenna signal, i.e. the coupling between the two RF antennas, is mainly affected by respiratory motion and less by cardiac motion. Since the first RF signal and the second RF signal are both complex, in fact four signals are present, wherein a blind source separation technique like PCA be applied to these four signals. In particular, a vector containing four elements can be defined, wherein each of the four elements corresponds to a respective one of the four signals. For example, each element of the vector can be a magnitude or phase of the first RF signal or the second RF signal or it can be the real component or the imaginary component of the respective RF signal. The blind source separation technique applied to this four-dimensional vector leads to a new four-dimensional vector in which the vector elements, i.e. the new subsignals, are uncorrelated, orthogonal or independent from each other, depending on the exact blind source separation technique used. Since cardiac motion, respiratory motion and other motion like bulk motion are mostly independent from each other, the vector elements of the new vector can be assigned to the different independent kinds of motion. The vector elements of the new vector, i.e. the corresponding new subsignals, can be regarded as being processed motion signals. In order to determine which processed motion signal is related to which type of motion, for a respective processed motion signal the value in a respective predefined expected frequency range, in which the respective motion is expected, can be compared with values outside of this frequency range. For instance, regarding cardiac motion, the expected frequency range can be 0.7 Hz to 1.5 Hz. The value of the respective processed motion signal in this frequency range can be compared with the values of the respective processed motion signal outside of this frequency range. The processed motion signal, for which the comparison yields the largest deviation between the value in the expected frequency range and the values outside of the expected frequency range, is regarded as being the processed motion signal that is related to the mechanical movement of the heart. This comparison can be, for instance, a division or subtraction. For instance, an average value for the expected frequency range can be compared with an average value outside of the expected frequency range, wherein this comparison can be carried out by dividing the two average values or by subtracting the two average values from each other.

In the same way, a second processed motion signal can be determined as being a processed motion signal that is caused by respiration, i.e. that is related to a mechanical movement of the lungs, wherein in this case the predefined expected frequency range can be, for instance, 0.15 Hz to 0.25 Hz. In an embodiment, a SOBI is used and the first SOBI component is the first processed motion signal that is related to the mechanical movement of the heart and the second SOBI component is the second processed motion signal that is related to the mechanical movement of the lungs. If the RF antenna module comprises several RF antennas like, for instance, 256 RF antennas, the measurement device can be configured to measure the coupling between the different RF antennas and also the reflection on the respective single RF antenna as the motion signals. In an embodiment, the measurement device is configured to measure the coupling between all RF antennas, i.e. for each pair of RF antennas a respective coupling is measured. The measured couplings between different RF antennas and the measured reflections on the respective single RF antenna can be used for forming a matrix with, for instance, 256×256 elements, if 256 RF antennas are used. This matrix, in particular the elements of the matrix, are time-dependent. The matrix can be used for combining the motion signals measured by the different RF antennas and for separating physiological motion components.

To measure the coupling between two RF antennas, an excitation signal can be provided to one of the two RF antennas while an RF signal is measured at the other of the two RF antennas. If the measured RF signal is complex, the measured RF signal has a phase and a magnitude. The measured RF signal is indicative of a scattering of the excitation signal provided to the one of the two RF antennas into the other of the two RF antennas and therefore is regarded as being the coupling. The RF instrument can be configured to carry out this measurement of the coupling for each pair of the several RF antennas. This measurement of the couplings can be carried out sequentially or simultaneously, wherein in the latter case several excitation signals with different frequencies are used, in order to allow to distinguish the measured RF signals, i.e. in order to determine by which excitation signal the respective scattered measured signal has been caused. Thus, a corresponding frequency demodulation is performed. After these couplings have been determined, for each RF antenna of the several RF antennas a respective reflection signal can be measured and for each combination of the respective RF antenna with the other RF antennas a corresponding coupling signal is present. In a preferred embodiment, these signals are complex such that for each reflection signal two respective subsignals are present and for each coupling signal also two respective subsignals are present. The processor can be configured to apply a blind source separation technique to these motion signals or subsignals, in order to determine processed motion signals. For instance, a SOBI can be applied and the first SOBI component can be a first processed motion signal and a second principal component can be a second processed motion signal. The RF instrument can be further configured to determine which processed motion signal corresponds to which type of motion by comparing the value of the respective motion signal in a respective predefined expected frequency range, in which the respective motion is to be expected, with values of the respective motion signal outside of this expected frequency range. In particular, in an embodiment such a comparison reveals that the first SOBI component is the processed motion signal that is related to the mechanical movement of the heart and the second SOBI component is the processed motion signal that is related to the mechanical movement of the lungs. In another embodiment, the comparison reveals that the first SOBI component is the processed motion signal that is related to the mechanical movement of the lungs and the second SOBI component is the processed motion signal that is related to the mechanical movement of the heart. In a further aspect of the present invention a measurement device is presented, wherein the measurement device is configured to be used with the determination device for forming the system for determining a physiological parameter, wherein the measurement device includes a) the RF antenna module comprising the one or more RF antennas and b) the RF instrument connected to the RF antenna module and configured to transmit RF power into the RF antenna module, to receive an RF signal from the RF antenna module and to provide a motion signal that is related to the mechanical movement of the structure within the subject. The measurement device can be configured to be worn by the subject.

In another aspect of the present invention a determination device for determining a physiological parameter of a subject based on a motion signal provided by the measurement device is presented, wherein the determination device comprises the model providing module configured to provide the model that provides, as an output, a physiological parameter if, as an input, a motion signal is provided, and the processor configured to determine the physiological parameter based on the provided model and the provided motion signal.

In a further aspect of the present invention a training system for training a model to be used by the system for determining the physiological parameter of the subject is presented, wherein the training system comprises:
a training physiological parameter measurement device for measuring a training physiological parameter of a subject,
a model providing module configured to provide an adaptable model to be trained, wherein the model provides, as an output, a physiological parameter if, as an input, a motion signal is provided,
an RF antenna module comprising one or more RF antennas and an RF instrument connected to the RF antenna module and configured to transmit RF power into the RF antenna module, to receive an RF signal from the RF antenna module and to provide a motion signal that is related to a mechanical movement of a structure within the subject based on the received RF signal, if the RF antenna module is arranged on the subject, and
a training module configured to a) determine a physiological parameter of the subject based on the model to be trained and a motion signal provided by the RF instrument and the RF antenna module and b) modify the model such that a deviation between the determined physiological parameter and the training physiological parameter is reduced.

Preferentially, the training physiological parameter measurement device is configured to use the RF antenna module for measuring the training physiological parameter of the subject. This allows to train the model and finally determine the physiological parameter with an even further increased accuracy, because the same RF antenna module can be used for determining the physiological parameter of the subject based on the model and the provided motion signal and for determining the training physiological parameter.

In another aspect of the present invention a method for determining a physiological parameter of a subject is presented, wherein the method comprises:
providing a motion signal that is related to a mechanical movement of a structure within the subject by using an RF instrument and an RF antenna module of a measurement device as defined by claim 13, providing a model that provides, as an output, a physiological parameter if, as an input, a motion signal is provided, by a model providing module, and
determining the physiological parameter based on the provided model and the provided motion signal by a processor.

In a further aspect of the present invention a training method for training a model to be used by the system for determining a physiological parameter of a subject, particularly as defined by any of claims 1 to 12, is presented, wherein the training method comprises:
providing a model to be trained by a model providing module, wherein the model provides, as an output, a physiological parameter if, as an input, a motion signal is provided,
measuring a training physiological parameter of a subject by a training physiological parameter measurement device and providing a motion signal that is related to a mechanical movement of an organ within a subject by using an RF antenna module comprising one or more RF antennas and an RF instrument connected to the RF antenna module and configured to transmit RF power into the RF antenna module, to receive an RF signal from the RF antenna module and to provide the motion signal based on the received RF signal, wherein the RF antenna module is arranged on the subject,
determining a physiological parameter of the subject based on the model to be trained and the motion signal provided by the RF instrument and the RF antenna module and modifying the model such that a deviation between the determined physiological parameter and the training physiological parameter is reduced by a training module.

In another aspect of the present invention a computer program for controlling a measurement device as defined by claim 13 is presented, wherein the computer program comprises program code means for causing the measurement device to provide a motion signal that is related to a mechanical movement of a structure within the subject by using an RF instrument and an RF antenna module of the measurement device. The computer program can be configured to run on the RF instrument or on a controller of the measurement device, which is configured to control the different components of the measurement device.

In a further aspect of the present invention a computer program for controlling a determination device for determining a physiological parameter as defined by claim 14 is presented, wherein the computer program comprises program code means for causing the determination device to determine the physiological parameter based on a provided model, which provides, as an output, a physiological parameter if, as an input, a motion signal is provided, and a motion signal which has been measured by a measurement device as defined by claim 13. The computer program can be configured to run on the processor of the determination device or on a controller of the determination device, which is configured to control the different components of the determination device.

In another aspect of the present invention a computer program for controlling a training system as defined by claim 15 is presented, wherein the computer program comprises program code means for causing the training system to carry out the steps of the training method, when the computer program is run on a computer controlling the training system. The computer program can be configured to run on one or several components of the training system defined in claim 15 or on a controller of the training system, which is configured to control the different components of the training system.

It shall be understood that the system for determining a physiological parameter of a subject of claim 1, the measurement device of claim 13, the determination device of claim 14, the training system of claim 15, the method for determining a physiological parameter of a subject of claim 17, the training method, the computer program for controlling a measurement device of claim 18, the computer program for controlling a determination device for determining a physiological parameter of claim 19, and the computer program for controlling a training system, have similar and/or identical preferred embodiments, particularly as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 shows schematically and exemplarily an embodiment of a system for determining a physiological parameter of a subject,

FIG. 2 shows schematically and exemplarily a measurement device of the system shown in FIG. 1, wherein the measurement device is configured to be worn by the subject,

FIG. 3 shows schematically and exemplarily an embodiment of a determination device of the system shown in FIG. 1, wherein the determination device is configured to determine the physiological parameter based on a motion signal provided by the measurement device shown in FIG. 2,

FIG. 4 shows schematically and exemplarily a training system for training a model to be used by the system shown in FIG. 1,

FIG. 5 shows schematically and exemplarily several signals provided by the measurement device for different operating frequencies,

FIG. 6 shows schematically and exemplarily a complex signal provided by the measurement device,

FIG. 7 shows schematically and exemplarily the provided signal after a phase rotation has been carried out,

FIG. 8 shows schematically and exemplarily the provided signal after a band-pass filter has been applied,

FIG. 9 shows a flowchart exemplarily illustrating an embodiment of a method for determining a physiological parameter of a subject,

FIG. 10 shows a flowchart exemplarily illustrating an embodiment of a training method for training a model to be used by the system for determining the physiological parameter of the subject,

FIG. 11 shows schematically and exemplarily a certain arrangement of RF antennas on a subject,

FIG. 12 shows schematically and exemplarily an embodiment of an RF antenna,

FIG. 13 shows schematically and exemplarily a further embodiment of an RF antenna,

FIG. 14 shows schematically and exemplarily a frequency spectrum of a motion signal measured by using the RF antenna shown in FIG. 13, and

FIG. 15 shows schematically and exemplarily the RF antenna shown in FIG. 13 placed on the chest of the subject.

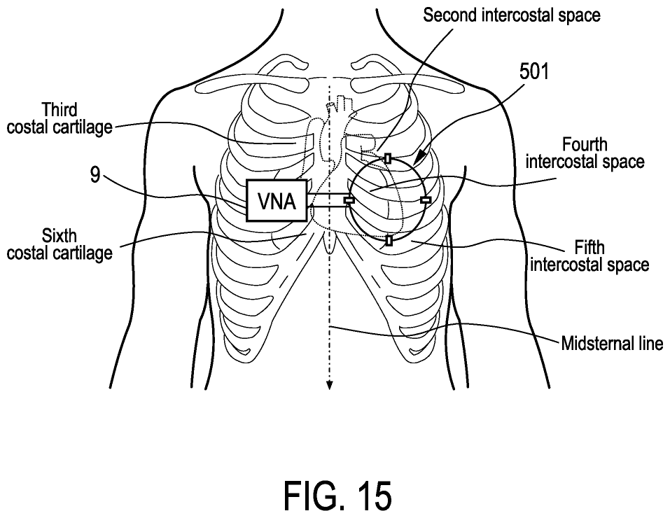

### DETAILED DESCRIPTION OF EMBODIMENTS

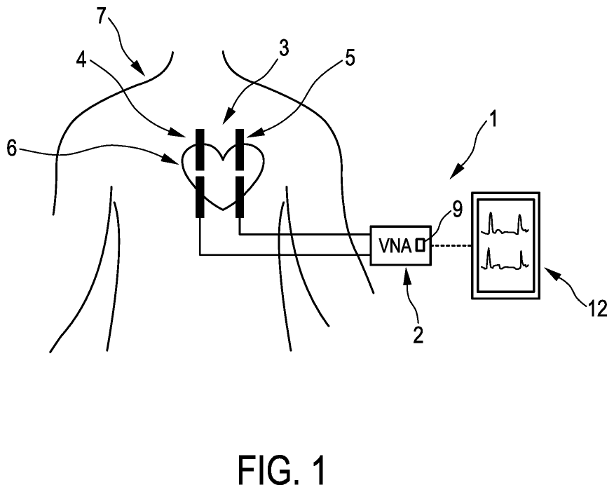

FIG. 1 illustrates schematically and exemplarily an embodiment of a system for determining a physiological parameter of a subject. In this embodiment the system 1 is configured to determine the stroke volume of the heart 6 within the subject 7 as the physiological parameter. However, the system also can be configured to determine another heart-related physiological parameter like the heart rate, or a lung-related physiological parameter like the respiratory rate.

The system 1 comprises a measurement device of which in FIG. 1 only two RF antennas 4, 5 of an RF antenna module 3 and a vector network analyzer 2 are shown. The RF antennas 4, 5 are connected to the vector network analyzer 2, wherein the vector network analyzer 2 and the RF antenna module 3, i.e. the RF antennas 4, 5, are configured to provide motion signals that are related to a mechanical movement of the heart 6 within the subject 7.

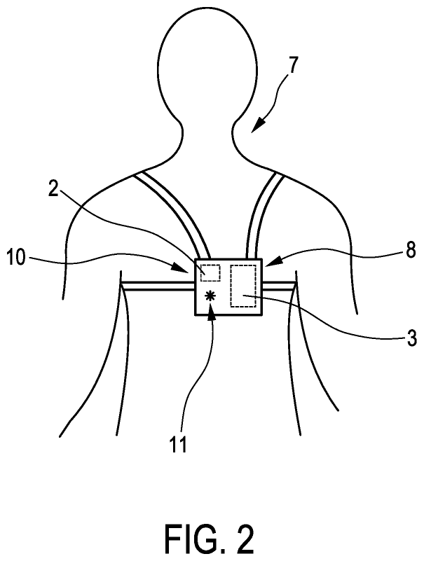

It should be noted that FIG. 1 just shows an illustration of the system 1 for determining a physiological parameter of the subject 7, wherein, for instance, the vector network analyzer 2 also is arranged on the body of the subject 7 and not somewhere in the air as shown in FIG. 1. In particular, as illustrated in FIG. 2, the measurement device 8 includes a wearable holder 10 in which the RF antenna module 3 and the vector network analyzer 2 are integrated as indicated schematically and exemplarily in FIG. 2 by dashed boxes.

The measurement device 8 further comprises a transmitter for transmitting the provided motion signals to a determination device 12 shown in FIG. 1. In this embodiment the transmitter 9 is integrated in the vector network analyzer 2 as schematically and exemplarily illustrated in FIG. 1. The transmission between the measurement device 8 and the determination device 12, particularly between the vector network analyzer 2 of the measurement device 8 and the determination device 12, is preferentially a wireless transmission like a Bluetooth transmission or any other wireless transmission.

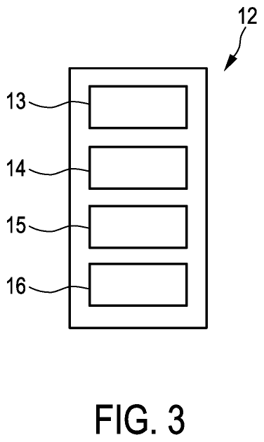

In this embodiment the determination device 12 is a smartphone or a tablet. The determination device 12 is configured to determine the physiological parameter, i.e. in this embodiment the stroke volume, based on the provided motion signals. The determination device 12 is exemplarily and schematically illustrated in FIG. 3.

The determination device 12 comprises a receiver 13 for receiving the motion signals provided by the measurement device 8. Moreover, the determination device 12 comprises a model providing module 14 configured to provide a model that provides, as an output, a physiological parameter, i.e. in this embodiment a stroke volume, if, as an input, the motion signals are provided. In this embodiment the model providing module 14 is a storage in which a correspondingly trained model is stored.

The determination device 12 further comprises a processor 15 configured to determine the physiological parameter based on the provided model and the received motion signals. The determination device 12 also comprises an output unit 16 like a display or a connector for connecting a display, in order to output the determined physiological parameter.

The wearable holder 10 comprises a visible marker 11 assigned to an anatomical feature of the subject 7, wherein the wearable holder 10 is configured to be worn such that the marker is arranged at a position on the subject 7 at which the assigned anatomical feature is located. In this embodiment the anatomical feature is the sternum at the height of the nipples, wherein, if the wearable holder 10 is worn correctly, the marker 11 coincides with the sternum at the height of the nipples.

The RF antenna module 3 has a width and a length, wherein the width and the length are each smaller than 20 cm. Moreover, in this embodiment the two RF antennas 4, 5 are dipole antennas. However, also other types of RF antennas could be used like loop coils.

The vector network analyzer 2 and the RF antenna module 3 are configured to be operated in a frequency range from 30 to 1000 MHz. In particular, the vector network analyzer 2 and the RF antenna module 3 are configured to be operated in a frequency range from 300 to 800 MHZ. In a preferred embodiment the operating frequency of the vector network analyzer 2 and the RF antenna module 3 is 64, 128 or 300 MHz.

The vector network analyzer 2 is configured to provide as a motion signal a complex signal like a complex reflection coefficient signal or complex coupling coefficient signal. In this embodiment, since the RF antenna module 3 comprises two RF antennas 4, 5, the vector network analyzer 2 provides two complex signals to the determination device 12.

The processor 15 of the determination device 12 is configured to identify, for each complex signal, a first subsignal of the respective complex signal having a distinct phase shift (for example 90 degrees) with respect to a second subsignal of the respective complex signal and to determine the physiological parameter based on the separated subsignals. In this embodiment the physiological parameter is the stroke volume, wherein for this reason it desired to have a subsignal which is related to the mechanical movement of the heart 6 within the subject 7, wherein an influence by other movements within the subject 7 should be as small as possible. It has been found that in the respective complex signal the contribution caused by cardiac motion has a distinct phase shift with respect to a contribution caused by respiratory motion. Thus, by identifying the phase shift of the first subsignal with respect to a second subsignal of the respective complex signal, the influence of respiratory motion on the signal, which is finally used for determining the stroke volume, can be strongly reduced or even eliminated.

The processor 15 can be further configured to apply a blind source separation technique to separate the subsignals out of the multiple complex signals received by the antenna, for example by applying ICA or SOBI. Furthermore, the processor 15 can be configured to apply a frequency filtering to the first subsignal like a band-pass filtering, a low-pass filtering, a high-pass filtering or Kalman filtering, in order to further reduce contributions to the first subsignal, which are not caused by the mechanical movement of the heart 6.

The resulting two first subsignals, which are obtained based on the two complex signals measured by using the two RF antennas 4, 5, can be combined to a combined signal by, for example, blind source separation like PCA. In another embodiment, the first subsignal with the least contribution from the second subsignal is selected based on spectral analysis. In particular, the processor can be configured to determine which first subsignal, i.e. which processed motion signal, has the largest deviation between a value in an expected frequency range, in which the respective motion is expected, and values outside of this frequency range. For instance, the processor can apply a Fourier transform for carrying out this comparison. The comparison can be between the expected frequency range and all values outside of the frequency range or the comparison can be between the expected frequency range and another unwanted frequency range in which an unwanted motion contribution is expected. For instance, if it should be determined which first subsignal, i.e. which processed motion signal, is related to cardiac motion, the expected frequency range can be 0.7 Hz to 1.5 Hz and the further unwanted frequency range with the unwanted motion being, for instance, respiratory motion can be 0.15 Hz to 0.25 Hz. The other way around, if it should be determined which motion signal is caused by respiratory motion, the expected frequency range being, in this case, for instance 0.15 Hz to 0.25 Hz can be compared with values in the in this case unwanted cardiac frequency range from, for instance, 0.7 Hz to 1.5 Hz or with all values outside of the expected frequency range. Thus, for instance, a peak value or average value in the expected frequency range can be compared with the average value or peak value in the unwanted frequency range or of all frequencies outside of the expected frequency range. The comparison can be carried out by division or subtraction as also described above. The first subsignal, for which the comparison provided the largest deviation to the values in the expected cardiac frequency range, is determined as being the processed motion signal that is related to mechanical movement of the heart. Correspondingly, the first subsignal, for which the comparison yields the largest deviation with respect to the respiration frequency range, is regarded as being the motion signal that is related to mechanical motion of the lungs. In other words, the first subsignal, for which the comparison resulted in a higher value in the expected cardiac frequency range, is regarded as being the cardiac processed motion signal and the first subsignal, for which the comparison resulted in a higher value in the expected the respiration frequency range, is regarded as being the respiration processed motion signal.

In this embodiment the model providing module 14 is configured to provide a linear model as the model. Thus, a linear function is provided, which relates the processed signal to the stroke volume. Correspondingly, the processor 15 is configured to determine the stroke volume based on the provided linear model and the provided processed signal, wherein the provided model has been determined before by training which also could be named calibration. An embodiment of a training system for training the model to be provided by the model providing module 14 will be described exemplarily in the following.

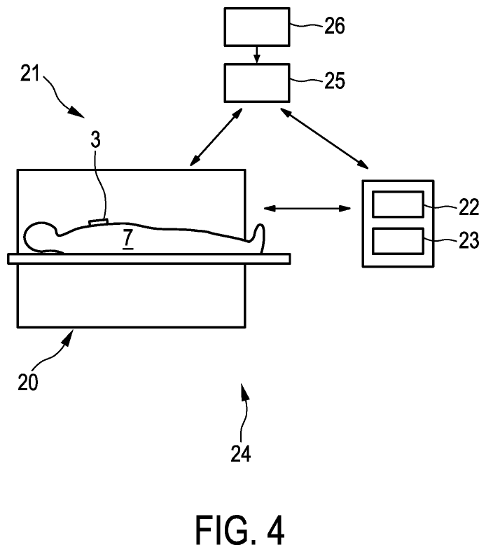

FIG. 4 shows schematically and exemplarily a training system 21 for training the model to be provided by the model providing module 14. The training system 21 comprises a training physiological parameter measurement device 24 for measuring a training physiological parameter of the subject 7 being, in this embodiment, the stroke volume.

The training physiological parameter measurement device 24 comprises an MR signals generation device 20 which uses the RF antenna module 3 of the measurement device 8 for determining the stroke volume. The training physiological parameter measurement device 24 further comprises a controller 22 for controlling the MR signals generation device 20 and a training physiological parameter determination module 23 for determining the training physiological parameter based on the generated MR signals. In this embodiment the training physiological parameter determination module 23 is configured to determine the stroke volume based on the MR signals generated by the MR signals generation device 20. For determining the stroke volume, the MR signals generation device 20, the controller 22 and the training physiological parameter determination module 23 can be configured to reconstruct MR images based on the MR signals and to determine the stroke volume based on the reconstructed MR images. In an embodiment, for determining the stroke volume, the MR signals generation device 20, the controller 22 and the training physiological parameter determination module 23 are configured to operate in accordance with known techniques like the technique disclosed in the article by Groepenhof et al., Physiological Measurements, 2007, 28 (1): 1-11 or in the article by Dornier et al., European Radiology, 2004 14 (8): 1348-52, which are herewith incorporated by reference. The MR signals generation device 20 can be a device of a standard MR system.

In another embodiment, also another training physiological parameter measurement device can be used. For example, the training physiological parameter measurement device can also be a Doppler echocardiographic device like the echocardiographic device disclosed in the article “Comparative accuracy of Doppler echocardiographic methods for clinical stroke volume determination” by Jonathan Dubin et al., American Heart Journal, volume 120, issue 1, pages 116 to 123 (1990), which is herewith incorporated by reference. In this case, the stroke volume is considered as the training physiological parameter. The training physiological parameter measurement device can also be a Fick device, a dye dilution device or a thermodilution device as described in the article “Thermodilution Cardiac Output: A Concept over 250 Years in Making” by E. Argueta et al., Cardiology in Review, volume 27, issue 3, pages 138 to 144 (2019), which is also herewith incorporated by reference. For this example, cardiac output is used as a training physiological parameter

The training system 21 further comprises a model providing module 26 configured to provide an adaptable model to be trained, wherein the model provides, as an output, a physiological parameter, if, as an input, a motion signal is provided. In this embodiment the model is a linear model of the type SV=ax+b, where x is, for example, the amplitude of the processed signal, the amplitude of the derivative of the processed signal, the area under a curve of the processed signal, the root-mean-square value or another quantity derived from the processed signal. SV is the stroke volume, which preferentially is defined as the volume of blood pumped per beat from the left ventricle, and a, b are adaptable parameters which are adapted during the training process. For instance, the processed signal is the one where the first subsignal is present most strongly in the frequency domain, i.e. has the largest amplitude, in other words, in an example, it is the above mentioned cardiac processed motion signal.

For determining the parameter “area under a curve of the motion signal”, the processor can be configured to detect peaks of the processed motion signal, in order to identify individual periods of the oscillating processed motion signal. For detecting the peaks known peak detection algorithms can be used like the algorithm disclosed in the article “A semi-automatic method for peak and valley detection in free-breathing respiratory waveforms” by W. Lu et al., Medical Physics, 33 (10): 3634-6 (2010), which is herewith incorporated by reference. The processor can be further configured to, for each peak-to-peak interval, integrate the total amplitude over time, in order to thereby determine the area under the curve. Thus, the respective part of the processed motion signal between two neighboring peaks is regarded as being the “curve”, wherein the integral value obtained by integrating the total amplitude over time between the two neighboring peaks is regarded as being the area under the respective curve. The total amplitude is defined as the difference between the maximum value and the minimum value of the respective curve.

In another embodiment, the model providing module can also be configured to provide another model like a Gaussian process regression model. As the Gaussian process regression model, a model as disclosed in the article “Gaussian processes for real-time 3D motion and uncertainty estimation during MR-guided radiotherapy” by N. Huttinga et al., Medical Image Analysis, ArXiv: 2204.09873 (2022) can be used. Also in this case, the model is used for mapping the processed signal to the physiological parameter being, for instance, the stroke volume or another physiological parameter like the ventricular movement speed.

In order to train the respective model like the Gaussian process regression model, in a training or calibration phase a reference physiological parameter, i.e. the gold standard, is compared with a physiological parameter determined by using the model to be trained. If the model is a Gaussian process regression model, a distribution of functions, which is associated with a mean and a covariance matrix, are modified, until the physiological parameter obtained by using the modified Gaussian process regression model corresponds as good as possible to the reference physiological parameter. For more details regarding the modification of the Gaussian process regression model, reference is made to the above-mentioned article by N. Huttinga et al.

Furthermore, the training system 21 comprises the measurement device 8 configured to be worn by the subject 7, wherein for clarity reasons in FIG. 4 of the measurement device 8 only the RF antenna module 3 is shown. As mentioned above, the training physiological parameter measurement device 24 and the measurement device 8 use the same RF antenna module 3.

The training system 21 further comprises a training module 25 configured to i) determine a physiological parameter of the subject 7 based on a) the model to be trained and b) a motion signal provided by the measurement device 8 by using the RF antenna module 3 and ii) modify the model such that a deviation between this determined physiological parameter and the training physiological parameter, which has been determined by the training physiological parameter measurement device 24 by using the same RF antenna module 3, is reduced, in particular, minimized. Preferentially, the training module 25 is configured to use the same processing of the signals, which is also applied by the processor 15 before the processor 15 uses the model as described above for determining the physiological parameter during an actual determination, i.e. after the training phase has been completed. In this embodiment the training model is configured to determine the stroke volume of the subject 7 based on the model to be trained, which is preferentially a linear model, and a processed signal which has been determined as described above and to modify the model such that a deviation between this determined stroke volume and a stroke volume determined by using the MR signals generation device 20, the controller 22 and the determination device 23 is reduced, particularly minimized.

The trained model is then used by the above described system 1 for determining a physiological parameter like the stroke volume of a subject. The system can perform dynamic determinations or measurements of the physiological parameter, in particular, of the cardiac output, with the one or more RF antennas integrated into the measurement device to be worn, particularly into the wearable holder. The system 1 can be used to monitor the pumping function of the heart at home in patients with heart failure.

Generally, RF antennas transmit RF electromagnetic radiation into the surrounding and receive electromagnetic radiation from the surrounding. An RF antenna has a measurable complex impedance which quantifies the relation between the complex current and voltage at a feed port of the respective RF antenna. This antenna impedance can be derived from RF reflection measurements with the vector network analyzer. The antenna impedance changes in phase and magnitude when the surrounding of the respective RF antenna changes. This happens when an RF antenna is positioned on the body and there is motion of the heart or the lungs. This effect can be utilized to measure internal physiological motion with RF antennas. It is noted that the RF operating frequency of the system determines the range in which physiological motion is detected. For example, when operating in a frequency range up to 300 MHZ, RF energy is absorbed in the whole body and global motion is measured. When operating in the range of 300 to 800 MHZ, motion of organs is measured, while higher frequencies can be used to measure local motion, just centimeters under the skin, for example, if the measurement device 8 is used to measure stroke volume of the heart, it does not use RF in the GHz range, but in a range of, for instance, 300 to 800 MHz.

RF antennas are used in MRI to excite nuclear spins and detect signals emitted back by the magnetized spins. Since the MRI antennas are also sensitive to physiological motion, they can be used to detect and correct for physiological motion in MRI. This is described, for instance, in the article “The rf coil as a sensitive motion detector for magnetic resonance imaging” by D. Buikman et al., Magnetic Resonance Imaging, volume 3, pages 281 to 289 (1988) which is herewith incorporated by reference. By operating at the same frequency as MRI antennas, during the above described training the same RF antennas can be used for determining, for instance, the stroke volume by using MRI and measuring a signal by using the measurement determination device 8, thereby allowing to use MRI as a unique calibration tool. Quantitative parameters like the stroke volume of the heart can be measured simultaneously with MRI and the measurement device 8. After this training or calibration, the physiological parameter of interest like the stroke volume can be measured by using the measurement device 8 only, wherein this measurement can even be carried out at home.

The operating frequency of the respective RF antenna determines the size of the area in which the RF energy is absorbed. Based on the wavelength of the RF radiation in tissue, power is deposited in the whole body or in parts of the body. For operating frequencies within the range of 30 to 300 MHz, the RF radiation causes power deposition throughout the whole body. For higher frequencies like 300 to 1000 MHz, power is deposited only in parts of the body, for example the head. For monitoring the full cardiorespiratory system, it can be beneficial to operate in the whole body resonant area like 30 to 300 MHz, or a frequency range in which resonance occurs only in the thorax. For monitoring smaller organs such as the heart, higher operating frequencies are desirable like 300 to 800 MHZ. For monitoring local motion of small structures, even higher operating frequencies like 800 to 1200 MHz are of interest. Generally, higher frequencies can be used to identify local motion of small structures, while lower frequencies can be used to identify motion of larger structures like full organs The used operating frequency therefore depends on the motion of which structure should be used for determining a respective physiological parameter. In the above described embodiment in which the stroke volume should be determined, the operating frequency is preferentially equal to the Larmor frequency of widely available MRI systems, i.e. 64 MHz for 1.5 T systems, 128 MHz for 3 T systems and 300 MHz for 7 T systems. This allows to use the same RF antenna for MRI and for the measurement carried out by the measurement device 8.

In an embodiment, RF frequency sweeps are performed, in which the operating frequency of the measurement device is changed over time. For example, by rapidly switching between measurements at 64 and 600 MHZ, information about whole body movement and organ movement can be obtained in a single measurement. When this is done, it should be ensured that the sample rate at the respective frequencies remains higher than two times the respective frequency to satisfy the Nyquist criterion.

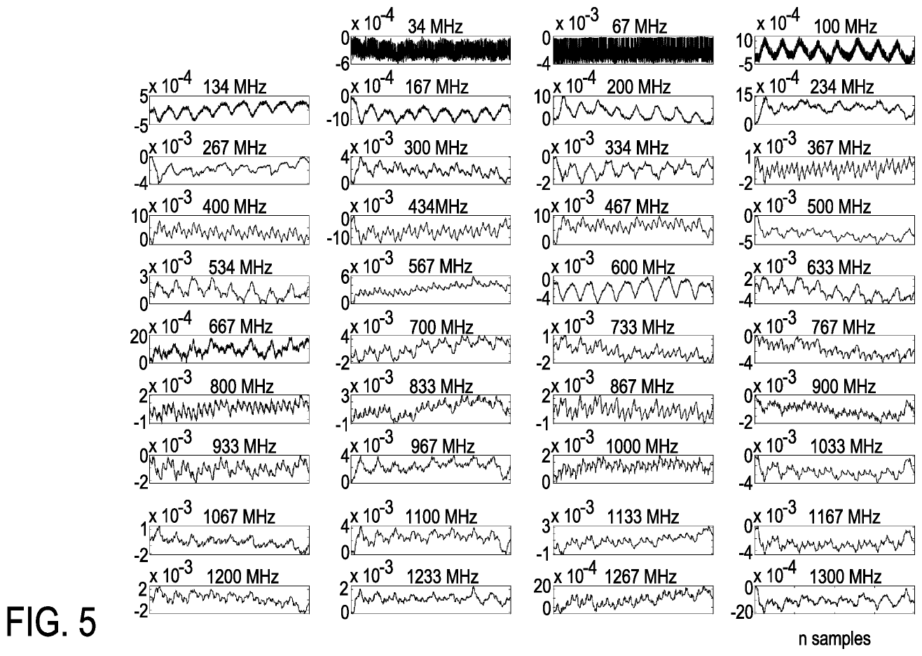

In an embodiment, the different frequencies cover a range from 30 MHz to 1300 MHz. For instance, the different frequencies can be 34 MHZ, 67 MHZ, 100 MHZ, 134 MHZ, 167 MHZ, 200 MHZ, 234 MHZ, 267 MHZ, 300 MHZ, 334 MHZ, 367 MHZ, 400 MHZ, 434 MHz, 467 MHz, 500 MHz, 534 MHZ, 567 MHZ, 600 MHZ, 633 MHz, 667 MHZ, 700 MHZ, 733 MHZ, 767 MHZ, 800 MHZ, 833 MHZ, 867 MHZ, 900 MHZ, 933 MHZ, 967 MHZ, 1000 MHz, 1033 MHz, 1067 MHZ, 1100 MHZ, 1133 MHZ, 1167 MHZ, 1200 MHZ, 1233 MHZ, 1267 MHz and 1300 MHz. Thus, the frequencies, at which the motion signal is measured, can be equidistantly distributed over the range of 30 MHz to 1300 MHz. Corresponding signals are schematically and exemplarily shown in FIG. 5. However, the different frequencies can also cover a smaller range like 30 MHz to 1000 MHz. Also if the frequency range is smaller than from 0 MHz to 1300 MHz, the different operation frequencies, at which the motion signal is measured, are preferentially equidistantly distributed over the frequency range.

The RF waves with different frequencies transmitted into the body have different penetration depths, different spatial sensitivity profiles and different spatial phase distributions. Movement of structures or parts of structures are therefore encoded differently in the different frequency components. Because of this, the signals acquired at different frequencies contain independent information on physiological motion.

In an embodiment the RF antennas can be loop antennas, which are commonly used in MRI, where they function as transmit and/or receive antennas. This enables simultaneous MRI and measurements with the measurement device.

Especially for measurements on the cardiorespiratory system, the processor is configured to achieve a separation of cardiac and respiratory signals, i.e. into a first subsignal being heart-related and a second subsignal being lung-related. Particularly if the measurement device is used for carrying out a complex reflection measurement, the resulting cardiac and respiratory signals are periodic and have a distinct phase difference, for example 90 degrees. In a preferred embodiment, the processor is configured to perform a phase rotation such that the cardiac signal, i.e. the first subsignal, appears on the real axis and the respiratory signal, i.e. the second subsignal, mostly on the imaginary axis. To be more generic, the processor can be configured to perform a transformation, in particular a 2×2 matrix transformation, on the complex signals measured by the measurement device 8 to achieve this.

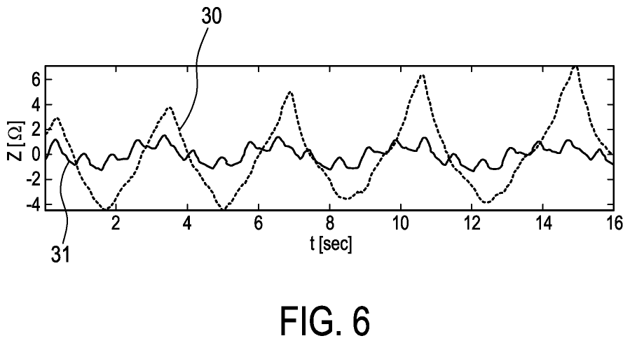

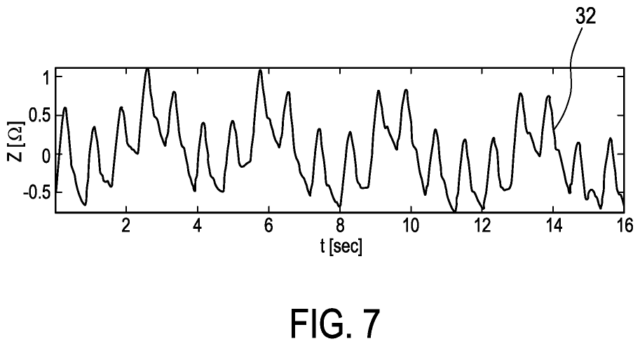

FIG. 6 schematically and exemplarily illustrates a measured complex signal Z over time, wherein in this figure the curve 30 is the imaginary part of the signal Z and the curve 31 is the real part of the signal Z. FIG. 7 illustrates the signal Z after the phase rotation has been carried out as described above, i.e. it shows the first subsignal along the real axis.

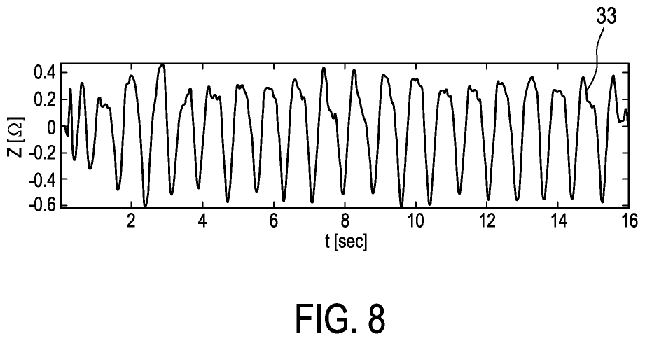

The processor can be further configured to exploit differences in spectral characteristics, in order to remove remaining contributions of motion that are not of interest. Thus, the processor can be configured to perform filtering in the frequency domain. For instance, a band-pass filter, a low-pass filter, a high-pass filter or a Kalman filtering could be used. In an embodiment, a band-pass filter between 0.75 and 10 Hz is used, in order to filter out remaining components of a respiratory signal. This is illustrated in FIG. 8. Thus, in FIG. 8 the curve 33 results from filtering the curve 32 shown in FIG. 7 by using a band-pass filter between 0.75 and 10 Hz.

A model can be used to predict stroke volume (SV, mL) from the measurement shown in FIG. 8. For example, if the amplitude of the signal, the amplitude of the derivative of the signal or any other quantity derived from the signal in FIG. 8 is given as x, the stroke volume can be calibrated through a linear relationship as SV=a*x+b, where a and b are determined in a calibration phase. In the calibration phase, the stroke volume is measured with a reference instrument such as MRI or ultrasound. This can be done under physiological stress, in order to increase the stroke volume during the measurements. At the same time, the parameter x is derived from the signal measured by using the RF antenna and the vector network analyzer. If SV and x are available for several different values of SV, the parameters a and b can be determined.

The above described system 1 can be used, for instance, to monitor the heart's pumping function in patients with heart failure at home. It can also be used to measure the heart rhythm and arrhythmia, or to quantify lung ventilation or edema, particularly to locally quantify lung ventilation or edema at home. For measuring the heart's pumping function, the stroke volume could be predicted based on a model of the effect of stroke volume on the measurements, for example the linear model SV=a*x+b. The same is possible for tidal volume, where the tidal volume (TV in mL) can be determined as TV=c*y+d, where y can be the amplitude of the respiration signal, and c and d are model parameters derived during a calibration measurement with a reference instrument such as spirometry. The TV and y can be measured during physiological stress, this will result in increasing TV over time. Based on this measurement, the parameters c and d can be determined. Parameters such as heart rate or respiratory rate can be derived from frequency domain analysis of the combined signals.

In another embodiment, the model providing module is configured to provide another model which provides the relation between the motion signal and the physiological parameter. For example, the model providing the relation between the stroke volume SV and the amplitude of the RF signal or the model providing the relation between the tidal volume TV and the amplitude y of the RF signal, which is related to breathing, could be a Gaussian process regression model like the Gaussian process regression model described in the above-mentioned article by Huttinga et al. The parameters of the Gaussian process regression model can be obtained in a training phase, wherein the parameters of the Gaussian process regression model are adapted such that the Gaussian process regression model outputs known given training physiological parameters, i.e. in this example known given SV or known given TV and optionally the uncertainty of the prediction, if, as an input, the amplitude of a respective RF signal is given.

It is also possible to track catheters during heart catheterizations and to monitor, for instance, heart-related and/or lung-related physiological parameters in sports.

In a further embodiment, the model providing module is configured to provide a model that provides, as an output, an echocardiography parameter if, as an input, the motion signal, i.e. in particular the RF signal, is provided. This model can be, for instance, a Gaussian process regression model. The echocardiography parameter is, for instance, the left ventricular outflow velocity. However, it can also be another echocardiography parameter. Echocardiography data being the left ventricular outflow velocity are described, for instance, in the article “Left ventricular outflow tract velocity time integral outperforms ejection fraction and Doppler-derived cardiac output for predicting outcomes in a select advanced heart failure cohort” by C. Tan et al., Journal of Cardiovascular Ultrasound, 15 (1): 18 (2017), which is herewith incorporated by reference. Also this model can be trained in a training phase, wherein the model is trained such that it outputs a known given echocardiography parameter like a known given left ventricular outflow velocity if, as an input, the RF signal is provided. As an input to the model, the motion signal is provided, which might be, for example, the time derivative of the RF signal, together with the echocardiography parameter that is obtained simultaneously as a reference. If a Gaussian process regression model is used, the model calculates during training a distribution of functions that explains the training data as good as possible, the distribution of functions can be characterized by mean and covariance parameters that are determined during training.

RF antennas can emit and detect electromagnetic radiation, wherein an RF impedance that is measured by an antenna changes based on the surrounding of the antenna. If an RF antenna is placed on a body and the dielectric properties of the body change, the RF impedance will also change. The dielectric properties of the body change during mechanical motion of, for example, the heart or the lungs, and they also change when external structures such as catheters are moving through the body. This enables the determination of, for instance, heart-related and/or lung-related physiological parameters based on motion of a structure of the subject like an organ or on motion of another structure like a catheter.

The RF antennas of the RF antenna module preferentially are flexible and lightweight, i.e. preferentially they have a weight being smaller than 30 g. In an embodiment, the RF antennas are integrated or even sewn in clothing. To measure RF back scattering with these RF antennas, the vector network analyzer is connected to the RF antennas. The vector network analyzer preferentially is a small mobile device which could even be held in a hand. The vector network analyzer is preferentially also integrated in the clothing. In particular, the above described wearable holder 10 does not only comprise the RF antennas of the RF antenna module, but also the vector network analyzer 2. However, in an embodiment it is also possible that the holder 10 only includes the RF antennas and that the vector network analyzer is held on the body by another means like a second holder.

As described above, the model can be trained such that it relates RF measurements carried out by the measurement device 8 to parameters obtained from MRI, wherein in the above described embodiment the physiological parameter is the stroke volume. To generate this model, a calibration step is performed, in which simultaneous MR imaging and RF measurements are done. Since the same one or several RF antennas are used for the MRI measurement and the RF measurement, the calibration can be integrated into an MRI process rather simply.

Although above a correlation with an output of an MRI measurement is described, the training or calibration could also be carried out with other kinds of measurements, i.e. with other calibration measurements, in order to correlate these other measurements like computed tomography (CT) or ultrasound measurements, i.e. physiological parameters obtained from these other measurements, to the RF measurements carried out by the measurement device which finally uses the correspondingly trained or calibrated model.

Generally, the model allows to provide a relation between a) the RF measurement carried out by using the measurement device, particularly the motion signal generated by using the RF measurement, and b) one or several physiological parameters obtained from an even relatively complex imaging modality like MRI or CT, wherein this relation can be used together with the RF measurements carried out by the measurement device, in order to determine a physiological parameter which normally would require a relatively complex imaging modality.

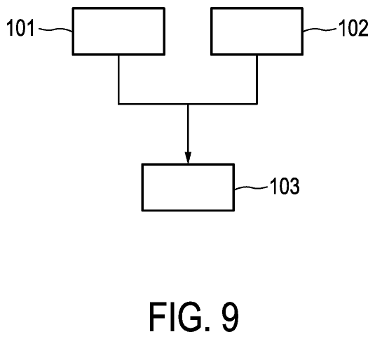

In the following an embodiment of a method for determining a physiological parameter of a subject will be described with reference to a flowchart shown in FIG. 9.

In step 101, a motion signal is provided, which is related to a mechanical movement of an organ like the heart within the subject by using a vector network analyzer and an RF antenna module of the measurement device 8. In step 102, a model is provided, wherein the model has been trained to provide, as an output, a physiological parameter if, as an input, a motion signal is provided. The model is provided by the model providing module 14. In step 103, the physiological parameter is determined based on the provided model and the provided motion signal by the processor 15.

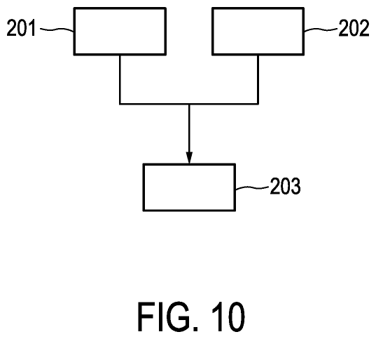

In the following an embodiment of a training method for training a model to be provided by the model providing module 14 will be exemplarily described with reference to a flowchart shown in FIG. 10.

In step 201, a training physiological parameter of a subject is measured by the training physiological parameter measurement device 24. For instance, by using MRI, a stroke volume of a heart is determined as the training physiological parameter. At the same time, a motion signal is provided, which is related to a mechanical movement of an organ within a subject, by using the measurement device 8. In particular, complex RF signals are measured, which are related to the mechanical movement of the heart. In step 202, a model to be trained is provided by a model providing module, wherein the model provides, as an output, a physiological parameter if, as an input, a motion signal is provided. In step 203, a physiological parameter of the subject is determined based on the model to be trained and the motion signal provided by the measurement device and the model is modified such that a deviation between the determined physiological parameter and the training physiological parameter is reduced, wherein this step is carried out by the training module 25. For instance, the model can be adapted such that a deviation between a stroke volume measured by the training physiological parameter measurement device 24 and a stroke volume determined by using the signal measured by the measurement device 8 and the model to be trained is reduced, particularly minimized.

Although in above described embodiments the RF antenna module has two RF antennas, the RF antenna module can also have a single RF antenna only or more than two RF antennas.

For instance, in an embodiment the RF antenna module comprises one RF antenna and the measurement device is configured such that, if the measurement device is worn by an adult, a center point of the RF antenna is positioned left to the sternum. This single RF antenna can be positioned with a shift within the range of 2 cm to 4 cm, further preferred with a shift of 3 cm, to the left of the sternum, in order to allow the measurement device to accurately measure a signal that is related to the movement of the heart.

If in another embodiment the RF antenna module comprises two RF antennas, these two RF antennas can be arranged such that they are positioned close to the heart, but at the same time positioned relatively far away from each other, in order to have a relatively low inter-element coupling. Such a configuration is schematically and exemplarily illustrated in FIG. 1. Thus, the two RF antennas can be arranged above the edges of the heart or above edge regions of the heart, wherein these edge regions could be left/right edge regions or top/down edge regions. Correspondingly, the distance between the two RF antennas is similar to a distance between these edge regions, wherein the dimensions of the heart and hence the distance between the edge regions can be determined beforehand by using an imaging modality like MRI or CT, or standard dimensions of a human heart of an adult or child, depending on the age of the subject, can be used for defining the arrangement of the two RF antennas.

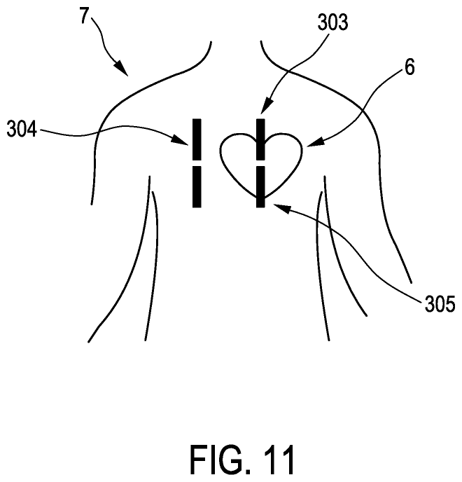

In a further embodiment, the RF antenna module 303 also comprises two RF antennas 304, 305, wherein the measurement device is configured such that, if the measurement device is worn by an adult, a center point of one 305 of the two RF antennas is positioned left to the sternum and a center point of the other 304 of the two RF antennas is positioned right to the sternum. This is schematically and exemplarily illustrated in FIG. 11. In particular, one of the RF antennas is positioned with a shift within the range of 2 cm to 4 cm, further preferred with a shift of 3 cm, to the left of the sternum and the other of the RF antennas is positioned with a shift within the range of 2 cm to 4 cm, further preferred with a shift of 3 cm, to the right of the sternum.

The holder can be anything which holds the RF antenna module on the body of the subject. It can be any wearable like a shirt, a band, et cetera, wherein especially for this reason the one or more RF antennas are preferentially flexible.

The vector network analyzer is portable and used to measure back scattering, i.e. the motion signal that is related to the mechanical movement of the organ like the heart. The measured signal preferentially is complex, i.e. it has a phase and a magnitude, wherein the vector network analyzer sends data representing the measured signal wirelessly to the determination device such as a personal computer or a mobile device, wherein the wireless data connection could be, for instance, Bluetooth or another wireless data connection.

The system for determining the physiological parameter of the subject can be configured to remotely monitor the heart function. For instance, heart failure can be monitored directly. Heart failure is a defect in the pumping function of the heart, for instance, the heart is not able to pump sufficient blood into the surrounding tissue which can lead to symptoms such as lung edema, sudden weight increase, tiredness and ultimate damage to the heart and other tissues. After a first treatment in a hospital, heart failure patients are very often re-hospitalized when symptoms of heart failure reoccur. Over 50% of all heart failure patients are re-hospitalized after six months of initial treatment. Heart failure is the leading cause of hospitalization in adults over 65 years in the U.S. Reoccurrence of heart failure is noticed when patients show symptoms, which is already too late, by then the function of the heart has deteriorated further. By using the above described system for determining a physiological parameter of a subject, it is possible to measure heart failure before symptoms occur, wherein the patient's medication or lifestyle then can be adapted to prevent re-hospitalization. With known systems it is not possible to measure the heart pumping function remotely and with enough accuracy. For example, a common remote measurement method for heart rhythm, electrocardiogrameasures only neurological impulses, but not the actual mechanical response of the heart to these impulses. ECG only measures heart rhythm, but not the heart pumping function. Since the above described system for determining a physiological parameter of a subject is sensitive to tissue deformation and changes in blood volume, the system can be used to sense changes in the heart pumping function, unlike ECG which is not directly sensitive to this.

The system also can be configured to monitor cardiac failure indirectly through detection of lung edema. Cardiac failure patients often suffer from lung edema as a result of cardiac failure. If the patients show symptoms of lung edema, there is already significant damage done to the heart and lungs. The system can be configured such that the provided motion signal is related to the mechanical movement of the lungs within the subject, wherein in this case the signal is very sensitive to respiratory motion. Since with developing lung edema the motion of the lungs changes, by monitoring the movement of the lungs, developing lung edema can be detected, thereby indirectly detecting cardiac failure. In this example, the determined physiological parameter can be a characteristic of the movement of the lungs like the frequency or amplitude of this movement.

If the system is configured to provide a motion signal that is related to a mechanical movement of the heart within a subject and to use this signal to determine a heart-related physiological parameter like the stroke volume or the heart rate, the heart-related physiological parameter can be used to monitor arrhythmia in cardiovascular patients. Such monitoring is normally done by using ECG measurements. However, ECG measurements use electrodes that are attached to the skin which is uncomfortable for patients. The above described system for determining a heart-related physiological parameter of the subject does not need to be attached to the skin, thus improving patient comfort.

The system can also be configured to remotely monitor lung ventilation. In particular, the measurement device can be configured to provide a motion signal that is related to the mechanical movement of the lungs within a subject, wherein the model can be trained such that, given the motion signal, a lung-related physiological parameter measured by, for instance, spirometry or MRI is output. The processor of the determination device then can determine a lung-related physiological parameter based on the provided motion signal and the trained model. In this case, for training the model a spirometry system or MRI system can be used.

The system can also be used for tracking catheters during implantation. During heart catheterization, generally a long thin tube is inserted in an artery or vein and threaded to the heart where it is used to treat or diagnose certain heart diseases. These catheters contain electrically conductive materials which makes RF measurements very sensitive to the position and movement of these wires. The resulting motion signal, which is related to the mechanical movement of the catheters, can be used for determining a physiological parameter like the stroke volume.

The system can also be configured to measure a heart-related physiological parameter like the heart rate or a lung-related physiological parameter like the breathing rate during physical exercise. It is known to do this with ECG which needs to make contact with the skin by using electrodes. In contrast to this, the above described system can measure the heart-related or lung-related physiological parameters without needing to make contact with the skin.

In a preferred embodiment in which the RF antenna module comprises two RF antennas only, the vector network analyzer and the two RF antennas can be configured such that an inter-element coupling between the two RF antennas is below a predefined value, wherein this predefined value might be, for instance, −12 dB. In particular, the distance between the two RF antennas is such that the inter-element coupling between the two RF antennas is below-12 dB. This distance can be, for instance, within a range of 4 to 8 cm and preferentially is 6 cm, wherein the distance refers to the distance between the center positions of the two RF antennas. The inter-element coupling might be measured with the two antennas and the RF instrument, which preferentially is a vector network analyzer, by quantifying the magnitude and phase of the signal that is reflected into antenna 2 when antenna 1 is transmitting.

In an embodiment the RF antenna module includes at least a first RF antenna and a second RF antenna, wherein the vector network analyzer and the RF antenna module are configured to measure a first motion signal of the first RF antenna that is related to a mechanical movement of a first organ within the subject and to measure a second motion signal of the second RF antenna that is related to a mechanical movement of a second organ within the subject, wherein the processor is configured to determine a coupling between the first RF antenna and the second RF antenna, to remove contributions of the movement of the second organ to the first motion signal from the first motion signal based on the determined coupling and to determine the physiological parameter based on the first motion signal. For instance, an antenna 1 can be positioned close to an organ of interest such that the reflection of antenna 1 is mainly affected by motion of this organ. An antenna 2 can be placed further away from the organ of interest and closer to another organ that can cause distortion in the signal of antenna 1, for example antenna 2 can be positioned closer to the lungs if the heart is of interest. The coupling between antenna 1 and antenna 2 will be more affected by motion of the organ that causes distortions, wherein the coupled signal from antenna 1 and 2 can be used to remove distortion from the signal of interest measured in antenna 1. To remove these artifacts, techniques such as blind source separation like SOBI can be used.

Moreover, in an embodiment, the RF antenna module includes several RF antennas having different transmit phases defining a sensitivity profile of the RF antenna module, wherein the RF antenna module is configured such that the sensitivity profile has its largest sensitivity at the location of the organ. For instance, the RF antenna module might comprise several RF antennas which are arranged in a belt-like configuration. The belt-like configuration can be such that the RF antennas are arranged around a torso of the subject. Preferentially, the number of RF antennas of this belt-like configuration is within a range from 3 to 32 RF antennas.

Although in above described embodiments the model mainly is a linear model, the model can also be another one. Generally, the model can be any relation between a) a physiological parameter like the stroke volume or a ventilation parameter and b) the motion signal provided by the measurement device. Such a relation could be determined by calibration/training, but also by electromagnetic simulation. For instance, for different distributions and dimensions of human components like organs, bones, skin, et cetera a respective electromagnetic simulation can be carried out and hence a respective relation, i.e. model, can be determined. Based on a specific distribution and specific dimensions of, for instance, the organs, the bones, the skin, et cetera of a respective subject, which might be known based on an image of the respective subject like an MRI, CT, ultrasound et cetera image, a matching model can be selected and used for determining the physiological parameter based on the motion signal. For carrying out the electromagnetic simulation, finite difference time domain simulations can be used. This can be done with commercially available electromagnetic solvers such as shown in the article by Navest et al., Magnetic Resonance in Medicine, 2019, 82:6 (2236-2247) which is herewith incorporated by reference.

In an embodiment, the relations and hence the models, which have been determined by electromagnetic simulation, together with body parameters describing the respective distributions and dimensions of human components like organs, bones, skin, et cetera can be used to train an artificial intelligence (AI). The body parameters could be, for instance, a dimension of the torso like its circumference and the AI can be trained such that, given one or several body parameters and the motion signal provided by the measurement device, the physiological parameter is output. Different AI methods could be used, for example regression models, Gaussian processes, neural networks, k-nearest neighbors or support vector machine. In an embodiment, scalar parameters such as body circumference, stroke volume at rest, BMI, age or sex are specified as input to the model. Moreover, in an embodiment, a model of the dielectric property distribution in the area of interest like the torso of the subject is obtained based on MRI, CT or ultrasound imaging. The dielectric property distribution can be provided as an input to train the AI and later to update the model.

In a further embodiment a specific distribution and specific dimensions of human components like organs, bones, skin, et cetera of the subject, of whom the relation between the motion signal provided by the measurement device and the physiological parameter should be determined, are determined based on an image of the subject like a CT or MR image, wherein the relation, i.e. the model, can be determined based on an electromagnetic simulation applied to the determined specific distribution and specific dimensions of the human components.

Although in above described embodiments the measurement device is configured to be worn by the subject, it is also possible that the measurement device is not configured to be worn by the subject. For instance, it can be a handheld device to be held in front of the subject like on the chest, in order to determine the physiological parameter. The measurement device can also be configured to be arranged on wall or to be arranged on a rack, stage or the like, wherein the subject can be arranged in front of the measurement device for determining the physiological parameter.

Although in above described embodiments, the one or several RF antennas have a certain construction, the one or several RF antennas can also be constructed in another way.

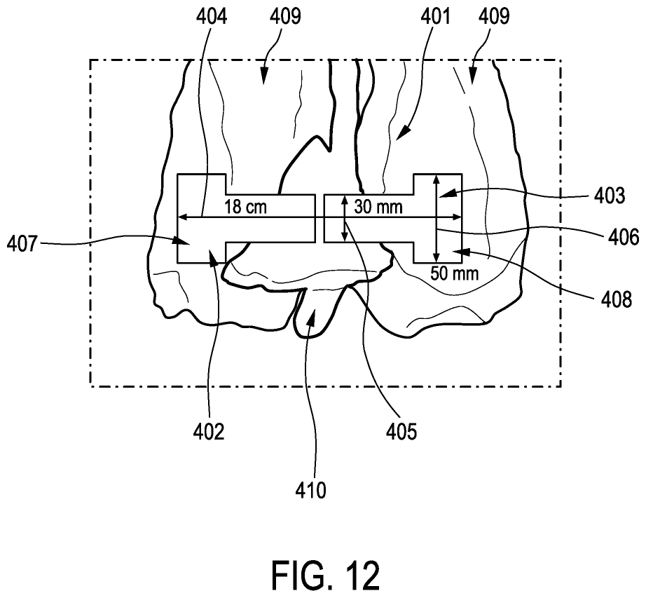

For instance, as illustrated in FIG. 12, an RF antenna 401 can be a dipole antenna with two separate conductive subelements 402, 403, i.e. the legs, having a T-like shape at their ends 407, 408 that face away from each other, i.e. having a T-like shape at their outer ends 407, 408. The T-like ends 407, 408 can increase the bandwidth and increase the sensitivity perpendicular to the longitudinal axis of the straight conductive element. It has been found by the inventors that a total antenna length 404 of 180 mm, i.e. a total length of the straight conductive element or, in other words, a length 404 from the end of one of the T-shapes to the end of the other of the T-shapes of 180 mm, a smaller width 405 of the respective T-like shape of 30 mm and a larger width 406 of the respective T-like shape of 50 mm provides an optimized tradeoff between sensitivity to cardiac motion and bandwidth. In a preferred embodiment, the dipole antenna 401 has these geometric dimensions or at least dimensions that are within 10% of the previously provided optimized values. The length direction of the dipole antenna 401 is defined by the longitudinal axis of the straight conductive element and the width direction is defined as being perpendicular to this longitudinal axis.

In FIG. 12, reference sign 409 indicates the lung and reference sign 410 indicates the heart. FIG. 12 illustrates how the dipole antenna 401 could be placed on the chest of the subject relative to the positions of the heart and the lung.

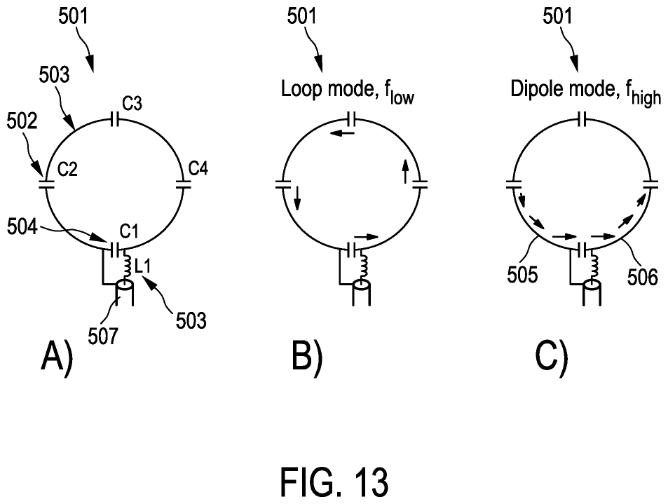

In a further embodiment, which is schematically and exemplarily shown in FIG. 13, the RF antenna is a loop coil 501 comprising a circular conductive element 503 like a correspondingly shaped wire or conductive strip, which has a gap 504 at which a matching circuit 503 and a connection to the RF instrument are placed. In this embodiment, the loop coil 501 has multiple further gaps 502 in its conductive element, wherein a respective capacitor C2, C3, C4 is placed in the respective further gap 502. Also in the gap 504, at which the matching circuit 503 is present, a capacitor C1 is placed.

The RF instrument can be configured to operate the loop coil 501 in a loop mode which is illustrated in FIG. 13B. The inductance of the conductive element in combination with the multiple capacitors makes the loop coil resonant at a respective frequency ω=1/\sqrt{LC}, wherein L is the total inductance of the conductive element, which might be circular, and C is the total capacitance of the multiple capacitors. The loop coil 501 is operated in the loop mode by using relatively low operation frequencies flow at which the capacitors do not act as blocking structures. Correspondingly, in the loop mode the current runs substantially uniformly along the whole length of the conductive element as indicated in FIG. 13B by the arrows.

The RF instrument can also be configured to operate the loop coil 501 in a dipole mode. In the dipole mode, the loop coil 501 acts like a dipole antenna. In the dipole mode the RF instrument uses an operation frequency f_{high} being so large that the current does not reach to capacitors C2 and C4. Correspondingly, in the dipole mode, only the subconductive elements 505, 506 connected to the RF instrument comprise current and these conductive subelements 505, 506, in which the current runs, act like a dipole antenna. This is indicated in FIG. 13C by the arrows.

By operating the loop coil in the loop mode and in the dipole mode, the loop coil can be resonant at multiple frequencies, thereby improving the sensitivity over a large frequency range, wherein this in turn can improve the accuracy of determining the physiological parameter.

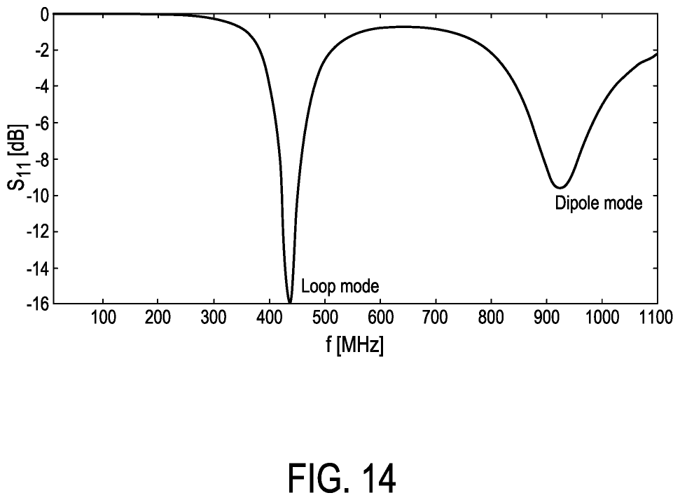

In a preferred embodiment, the diameter of the circular conductive element 503 of the loop coil 501, for instance, the diameter of a circular conductive element 503 of the loop coil 501, is within a range from 70 mm to 120 mm, further preferred within a range from 100 mm to 120 mm and most preferentially it is 110 mm. It has been found by the inventors that a loop coil with a diameter with these values allows to provide a motion signal that is related to a mechanical movement of the heart with a further increased accuracy. Moreover, in a preferred embodiment the loop coil is tuned to 433 MHz as a first resonance frequency and to 920 MHz as a second order resonance frequency, as schematically and exemplarily illustrated in FIG. 14. These frequencies correspond to radio bands that are reserved for industry, medical and scientific (ISM) purposes.

The matching circuit 503 can comprise a matching inductor L1 or alternatively a capacitor and a lattice balun to match the impedance of the loop coil 501 to the characteristic impedance of the connection 507 to the RF instrument, which preferentially is a coaxial cable.

In an embodiment, the loop coil has a diameter of 110 mm, the capacitors C2, C3 and C4 each have a capacitance value of 1.8 pF, the capacitor C1 has a capacitance value of 5.6 pF and the matching inductor L1 has an inductance value of 22 nH. These values are particularly preferred, if the loop coil should be tuned to 433 MHZ (loop mode) as a first resonance frequency and to 920 MHZ (dipole mode) as a second order resonance frequency.

In a further embodiment, the loop coil is operated with an operating frequency of 140 MHZ, it has a diameter of 110 mm, the capacitors C2-C4 have capacitance values of 15 pF, the capacitor C1 has a capacitance value of 33 pF and the inductor L1 has a value of 82 nH.

In a preferred embodiment, the loop coil 501 is arranged such on the subject's chest that the capacitor C1 in the gap 504 close to the connection to the RF instrument can be placed at the midsternal line, in particular at the height of the fourth intercostal space. In particular, the loop coil can be arranged in a wearable holder, wherein the wearable holder and the loop coil 501 can be arranged such that the capacitor C1 close to the connection to the RF instrument is placed at the center of the midsternal line, preferentially at the height of the fourth inter-coastal space, if the wearable holder is worn by the subject.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the determination of the physiological parameter, the training of the model, et cetera performed by one or several units or devices can be performed by any other number of units or devices. These procedures and/or the control of the components of the system for determining the physiological parameter of the subject in accordance with the above described method for determining the physiological parameter of the subject and/or the control of the training system in accordance with the training method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A system for determining a physiological parameter of a subject, the system comprising:
a measurement device including:
a) an RF antenna module comprising one or more RF antennas; and
b) an RF instrument connected to the RF antenna module and configured to transmit RF power into the RF antenna module, to receive an RF signal from the RF antenna module and to provide a motion signal that is related to a mechanical movement of a structure within the subject based on the received RF signal;
a determination device configured to determine the physiological parameter based on the provided motion signal, wherein the determination device comprises a model providing module configured to provide a model that provides, as an output, a physiological parameter if, as an input, a motion signal is provided; and
a processor configured to determine the physiological parameter based on the provided model and the provided motion signal.

2. The system of claim 1, wherein the RF instrument is configured to provide as the motion signal a complex signal.

3. The system of claim 2, wherein the processor is configured to identify a first subsignal of the complex signal having a distinct phase shift with respect to a second subsignal of the complex signal and to determine the physiological parameter based on at least one of the subsignals, wherein preferentially the phase shift is 90 degrees.

4. The system of claim 1, wherein the RF instrument and the RF antenna module are configured to be operated in a frequency range from 30 to 1000 MHz, further preferred in a frequency range from 300 to 800 MHz and even further preferred in a frequency range from 30 to 300 MHz.

5. The system of claim 4, wherein the RF instrument and the RF antenna module are configured to be operated with an operating frequency of 64, 128 or 300 MHz.

6. The system of claim 1, wherein the RF antenna module includes at least a first RF antenna and a second RF antenna, wherein the RF instrument and the RF antenna module are configured to provide a first motion signal of the first RF antenna that is related to a mechanical movement of a first structure within the subject and to measure a second motion signal of the second RF antenna that is related to a mechanical movement of a second structure within the subject, wherein the processor is configured to remove contributions of the movement of the second structure to the first motion signal from the first signal based on the provided first and second motion signals and to determine the physiological parameter based on the first signal.

7. The system of claim 1, wherein the processor is configured to apply a blind source separation technique, in order to generate a processed motion signal, and to use the processed motion signal and the provided model to determine the physiological parameter, and/or wherein the model providing module is configured to provide as the model at least one of a linear regression model, a polynomial regression model, and a Gaussian process regression model.

8. The system of claim 7, wherein the processor is configured to apply a second order blind identification (SOBI) as the blind source separation technique, thereby generating a SOBI component as the processed motion signal, and wherein the model providing module is configured to provide as the model the Gaussian process regression model.

9. The system of claim 1, wherein the RF antenna module includes several RF antennas having different transmit phases defining a sensitivity profile of the RF antenna module, and wherein the RF antenna module is configured such that the sensitivity profile has its largest sensitivity at the location of the structure.

10. The system of claim 1, wherein the measurement device is configured to measure different motion signals for different frequencies, and wherein the determination device is configured to determine the physiological parameter based on the motion signals measured for the different frequencies.

11. The system of claim 1, wherein the one or more RF antennas include at least one of a dipole antenna with a gap and a loop coil with a gap in which a capacitor is arranged.

12. The system of claim 11, wherein the one or more RF antennas include a loop coil comprising a conductive element with multiple gaps, wherein a respective capacitor is placed in a respective gap, and wherein preferentially the RF instrument is configured to operate the loop coil in a loop mode, in which the current runs along the whole length of the conductive element, and/or in a dipole mode in which the loop coil acts like a dipole antenna.

13. A measurement device configured to be used with a determination device for forming the system for determining a physiological parameter of claim 1;
wherein the determination device is configured to determine a physiological parameter of a subject based on a motion signal measured by the measurement device, wherein the determination device comprises a model providing module configured to provide a model that provides, as an output, a physiological parameter if, as an input, a motion signal is provided, and a processor configured to determine the physiological parameter based on the provided model and the provided motion signal;
wherein the measurement device includes; a) an RF antenna module comprising one or more RF antennas, and b) an RF instrument connected to the RF antenna module and configured to transmit RF power into the RF antenna module, to receive an RF signal from the RF antenna module and to provide a motion signal that is related to a mechanical movement of a structure within the subject.

14. A determination device for determining a physiological parameter of a subject based on a motion signal measured by the measurement device of claim 13, wherein the determination device comprises a model providing module configured to provide a model that provides, as an output, a physiological parameter if, as an input, a motion signal is provided, and a processor configured to determine the physiological parameter based on the provided model and the provided motion signal.

15. A training system for training a model to be used by a system for determining a physiological parameter of a subject as recited in claim 1, wherein the training system comprises:
a training physiological parameter measurement device for measuring a training physiological parameter of a subject;
a model providing module configured to provide an adaptable model to be trained, wherein the model provides, as an output, a physiological parameter if, as an input, a motion signal is provided;
an RF antenna module comprising one or more RF antennas and an RF instrument connected to the RF antenna module and configured to transmit RF power into the RF antenna module, to receive an RF signal from the RF antenna module and to provide a motion signal that is related to a mechanical movement of a structure within the subject based on the received RF signal, if the RF antenna module is arranged on the subject; and
a training module configured to:
a) determine a physiological parameter of the subject based on the model to be trained and a motion signal provided by the RF instrument and the RF antenna module; and
b) modify the model such that a deviation between the determined physiological parameter and the training physiological parameter is reduced.

16. The training system of claim 15, wherein the training physiological parameter measurement device is configured to use the RF antenna module for measuring the training physiological parameter of the subject.

17. A method for determining a physiological parameter of a subject, the method comprising:
providing a motion signal that is related to a mechanical movement of a structure within the subject by using an RF instrument and an RF antenna module of a measurement device;
providing a model that provides, as an output, a physiological parameter if, as an input, a motion signal is provided, by a model providing module; and
determining the physiological parameter based on the provided model and the provided motion signal by a processor.

18. A computer program for controlling a measurement device of claim 13, wherein the computer program comprises program code means for causing the measurement device to provide a motion signal that is related to a mechanical movement of a structure within the subject by using an RF instrument and an RF antenna module of the measurement device.

19. A computer program for controlling a determination device for determining a physiological parameter of claim 14, wherein the computer program comprises program code means for causing the determination device to determine the physiological parameter based on a provided model, which provides, as an output, a physiological parameter if, as an input, a motion signal is provided, and a motion signal which has been provided by a measurement device which includes; a) an RF antenna module comprising one or more RF antennas, and b) an RF instrument connected to the RF antenna module and configured to transmit RF power into the RF antenna module, to receive an RF signal from the RF antenna module and to provide a motion signal that is related to a mechanical movement of a structure within a subject.
